(19) United States
(12) Patent Application Publication
(10) Pub. No.: US 2026/0286375 A1
(43) Pub. Date: Sep. 24, 2026

(54) METHOD OF PRODUCING STYRYLPYRONES AND CHALCONES

(71) Applicant: NICOVENTURES TRADING LIMITED, London (GB)

(72) Inventors: Omer BAYAZEID, Winchester (GB); Anupriya THIND, London (GB); Sara BEN KHALED, Southampton (GB)

(21) Appl. No.: 19/478,137

(22) PCT Filed: Apr. 26, 2024
(86) PCT No.: PCT/GB2024/051090

§ 371 Date: Oct. 23, 2025

(30) Foreign Application Priority Data
Apr. 28, 2023 (GB) 2306343.1

(51) Int. Cl. C12N 15/82; C07K 14/415; C12N 9/10; C12N 15/52

(52) U.S. Cl. CPC C12N 15/8243; C07K 14/415; C12N 9/1007; C12N 9/1037; C12N 15/52; C12N 15/8205; C12Y 203/01074

(57) ABSTRACT

The present disclosure relates to a composition, cells transformed with the composition, use of the composition, methods of using the composition, and products obtainable from the method.

### FIELD OF THE INVENTION

The present disclosure relates to a composition, cells transformed with the composition, use of the composition, methods of using the composition, and products obtainable from the method.

### BACKGROUND OF THE INVENTION

In 2021, the global dietary supplements market size was valued at USD 151.9 billion, with an expected compound annual growth rate of 8.9% from 2022 to 2030 (Grand View Research. Report ID: 978-1-68038-919-7. Retrieved from https://www.grandviewresearch.com/industry-analysis/dietary-supplements-market). Of this, the global kava root extract market size was valued at USD 1.02 billion with an expected compound annual growth rate of 16.29% between 2022 and 2029 (Fortune Business Insights. Report ID: FBI103694. Retrieved from https://www.fortunebusinessinsights.com/kava-root-extract-market-103694).

Kava is a plant native to the Polynesian islands, where Polynesian indigenous cultures have prepared its roots as a beverage for use in religious and cultural rituals for generations. In some traditions, kava beverages are consumed as a social drink, similar to the consumption of alcoholic beverages. The popularity of kava is growing, mostly due to its relaxing properties and therapeutic effects, including anxiolytic and analgesic properties. Therefore, kava is also now utilised in various pharmaceutical and personal care products, typically in the form of capsules or tablets.

The bioactive ingredients of kava are styrylpyrone derivatives termed kavalactone or kavapyrone compounds, including yangonin, desmethoxyyangonin, kavain, dihydrokavain, methysticin and dihydromethysticin, which have a significant effect on several neurotransmitter systems including glutamine, γ-aminobutyric acid (GABA), dopamine and serotonin, leading to the exhibition of anxiolytic, analgesic, or neuroprotective effects by kavalactones (Bian et al. Kava as a Clinical Nutrient: Promises and Challenges. Nutrients 2020, 12, 3044). Other bioactive ingredients of kava are flavokavains also known as flavokawains, which are chalcones, and include flavokavains A, B and C.

Kavalactones are a subgroup of the styrylpyrone group that originates from phenylpropanoid natural products found in plants and fungi (Wu et al. De novo biosynthesis of diverse plant-derived styrylpyrones in Saccharomyces cerevisiae. Metab Eng Commun. 2022 March 5; 14:e00195; Pluskal et al. The biosynthetic origin of psychoactive kavalactones in kava. Nat. Plants. 2020, 5, 867-878). Styrylpyrone compounds share a common styrylpyrone scaffold/backbone, which is formed by two condensation reactions, followed by a ring closure (lactonization) of the triketide intermediates to the styrylpyrones, catalysed by a polyketide synthase (Schröder, J. 1.27—The Chalcone/Stilbene Synthase-type Family of Condensing Enzymes, Eds: Barton, et al. Comprehensive Natural Products Chemistry, Pergamon, 1999, 749-771). The styryl moiety, derived from phenylalanine via cinnamic acid, p-coumaric acid and caffeoylcoenzyme-A and pyrone ring, is acetate derived. On the other hand, flavokavains are a subgroup of the chalcones group and are formed through Claisen condensation of triketide intermediates.

In addition to the bioactive properties of kavalactones listed above, other plant derived styrylpyrones, such as 7,8-dihydro-5,6-dehydrokavain isolated from shell ginger, also exhibit bioactive properties. In addition, styrylpyrones and/or chalcones isolated from medicinal fungi have shown anti-oxidative, anti-cancer, anti-platelet, anti-diabetic, anti-inflammatory and antiviral activities. Thus, styrylpyrones and/or chalcones present two diverse classes of bioactive compounds with potential nutritional and pharmaceutical uses.

### SUMMARY OF THE INVENTION

In one aspect, there is provided a composition for promoting the expression of one or more styrylpyrones and/or chalcones in a cell, wherein the composition comprises:
a) one or more first vector components encoding one or more enzymes present in the kavalactone or flavokavain pathways; and
b) one or more second vector components encoding one or more transcription factors present in the flavonoid pathway.

In one embodiment, the one or more first vector components are provided in a first vector and the one or more second vector components are provided in a second vector.

In an alternative embodiment, the one or more first vector components and the one or more second vector components are provided in a single vector.

In one aspect, there is provided a plant, plant part or plant cell transformed with the vector components according to any one of the composition embodiments described herein.

In one aspect, there is provided use of the composition according to any one of the composition embodiments described herein for promoting the expression of one or more styrylpyrones and/or chalcones in algae, moss, or any plant, plant part, or plant cells.

In one aspect, there is provided a method of promoting the expression of one or more styrylpyrones and/or chalcones in a cell, comprising the step of:
a) introducing the composition according to any one of the composition embodiments described herein into a cell.

Optionally, the method further comprises the steps of:
b) extracting the one or more styrylpyrones and/or chalcones from the cell to produce an extract;
c) detecting the one or more styrylpyrones and/or chalcones in the extract; and
d) purifying the one or more styrylpyrones and/or chalcones from the extract.

In one aspect, there is provided a styrylpyrone or chalcone obtainable from the method according to any one of the method embodiments described herein.

### BRIEF DESCRIPTION OF THE DRAWINGS

Embodiments of the invention will now be described, by way of example only, with reference to accompanying drawings, in which:

FIGS. 1A and 1B show HPTLC results for combinations of SPS1, SPS2, MYB12, DEL and ROS1 against a yangonin standard. KOMT1 was present in all combinations (excluding the yangonin standard). Stationary Phase; Merck, HPTLC Silica gel 60 F₂₅₄, Mobile phase; Hexane:Dioxen 8:2 (v/v). All samples dissolved in Methanol.

FIG. 2 schematically shows a customized pCAmbia2300 vector encoding AmDEL, AmROS1 and AtMYB12 for plant cell lines and Nicotiana species.

FIG. 3 schematically shows a customized pCAmbia2300 vector encoding KOMT1 and SPS1 for plant cell lines and Nicotiana species.

FIG. 4 schematically show a customized pCAmbia2300 vector encoding AmDEL and AmROS1 for plant cell lines and Nicotiana species.

FIG. 5 schematically shows a customized pMBL5DL108 vector encoding KOMT1 and SPS1 for moss.

FIG. 6 schematically shows a customized pCAmbia2300 vector encoding AtMYB12 for plant cell lines and Nicotiana species.

FIG. 7 schematically shows a customized CAt203 vector encoding SPS1, KOMT1, AmDEL, AtMYB12 and AmROS1 for plant cell lines and Nicotiana species.

FIG. 8 schematically shows the synthesis pathway for styrylpyrones, chalcones, kavalactones and flavokavains. Enzymatic activity is indicated by arrows. Phenylalanine ammonia-lyase (PAL), 4-coumarate:CoA ligase (4CL), cinnamate-CoA ligase (CNL), styrylpyrone biosynthesis (SPS), chalcone synthase (CHS), kava O-methyltransferase 1 (KOMT1).

FIGS. 9A-C schematically show the synthesis pathway for styrylpyrones and kavalactones. Enzymatic activity is indicated by arrows. FIG. 9A shows the phenylpropanoid pathway which provides the precursor compounds for styrylpyrone synthesis. FIG. 9B shows the conversion of phenylpropanoids to styrylpyrones. FIG. 9C shows the conversion of styrylpyrones to kavalactones. Phenylalanine ammonia-lyase (PAL), cinnamate 4-hydroxylase (C4H), 4-coumarate:CoA ligase (4CL), hydroxycinnamoyltransferase (HCT), p-coumaroyl shikimate 3′-hydroxylase (C3′H), caffeoyl shikimate esterase (CSE), caffeic acid O-methyltransferase (COMT), caffeoyl-CoA O-methyltransferase (CCoAOMT), coumarate 2-hydroxylase (C2H), cinnamate-CoA ligase (CNL), styrylpyrone biosynthesis 1 (SPS1), styrylpyrone biosynthesis 2 (SPS2), chalcone synthase (CHS), kava O-methyltransferase 1 (KOMT1), kava O-methyltransferase 2 (KOMT2), kavalactone reductase 1 (KLR1), P450, and CYP719A26.

### BRIEF DESCRIPTION OF THE SEQUENCES

A summary of sequence identifiers used throughout the subject specification and the corresponding sequence listing is provided, wherein:

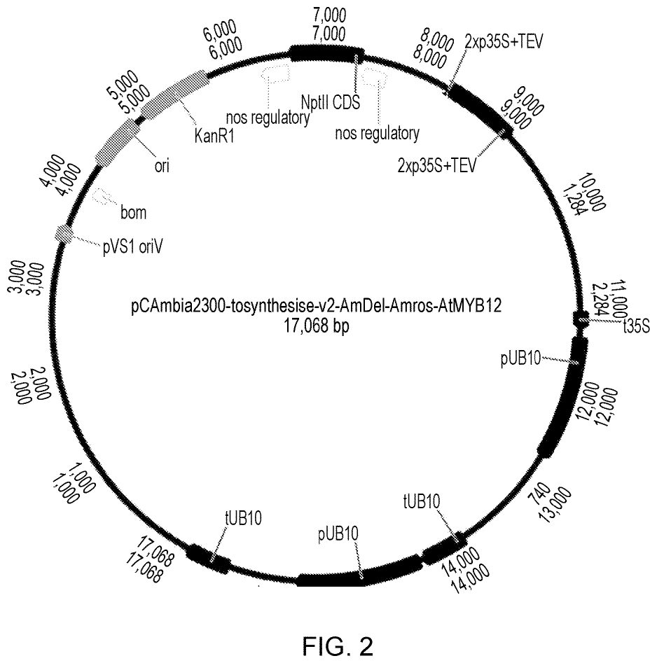

SEQ ID NO. 1 corresponds to the nucleic acid sequence of the pCAmbia2300 vector of FIG. 2, which encodes AmDEL, AmROS1 and AtMYB12.

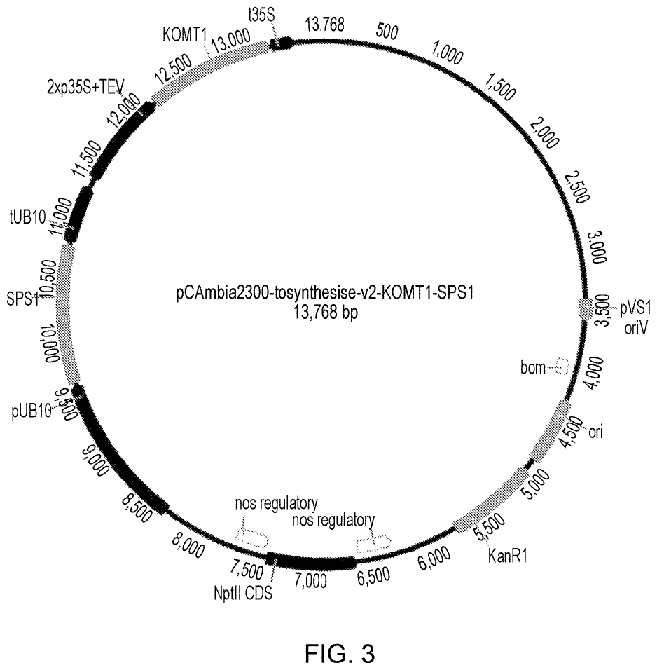

SEQ ID NO. 2 corresponds to the nucleic acid sequence of the pCAmbia2300 vector of FIG. 3, which encodes KOMT1 and SPS1.

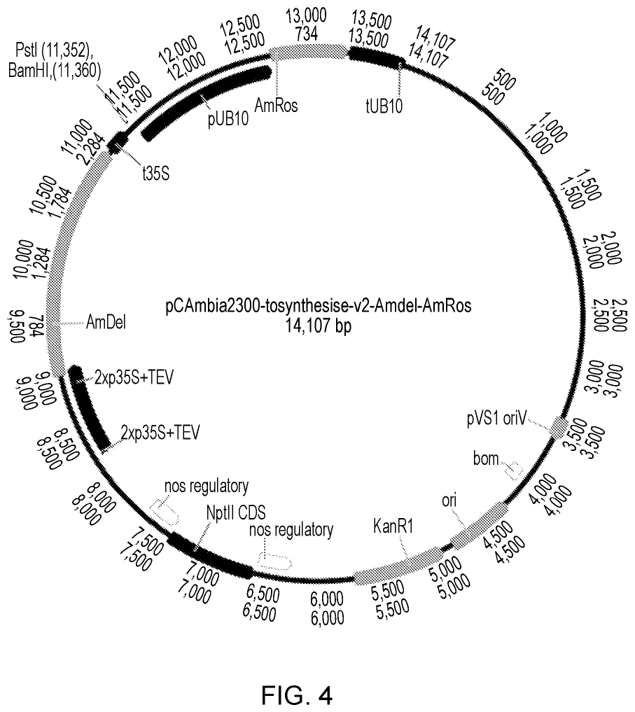

SEQ ID NO. 3 corresponds to the nucleic acid sequence of the pCAmbia2300 vector of FIG. 4, which encodes AmDEL and AmROS1.

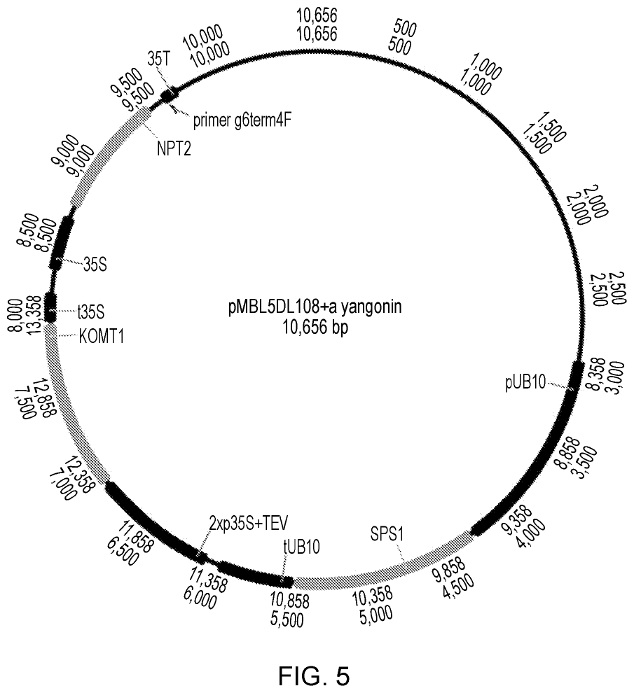

SEQ ID NO. 4 corresponds to the nucleic acid sequence of the pMBL5DL108 vector of FIG. 5, which encodes KOMT1 and SPS1.

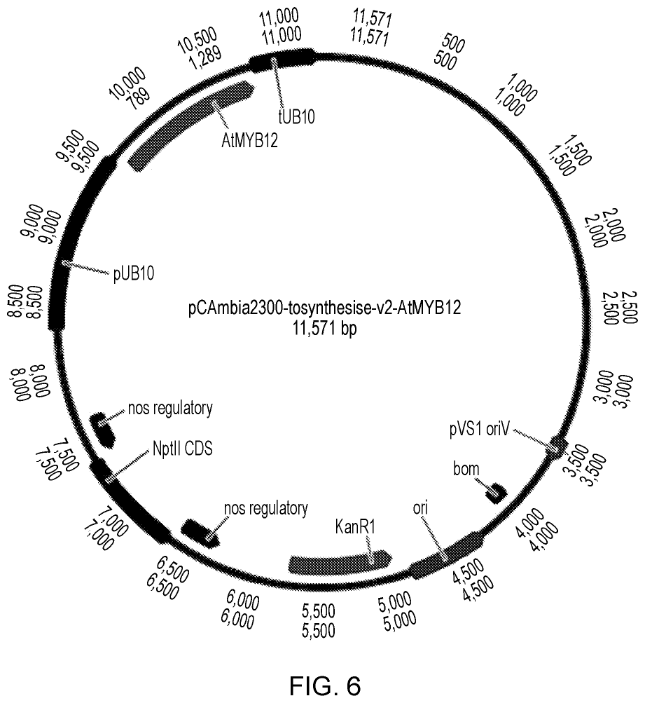

SEQ ID NO. 5 corresponds to the nucleic acid sequence of the pCAmbia2300 vector of FIG. 6, which encodes AtMYB12.

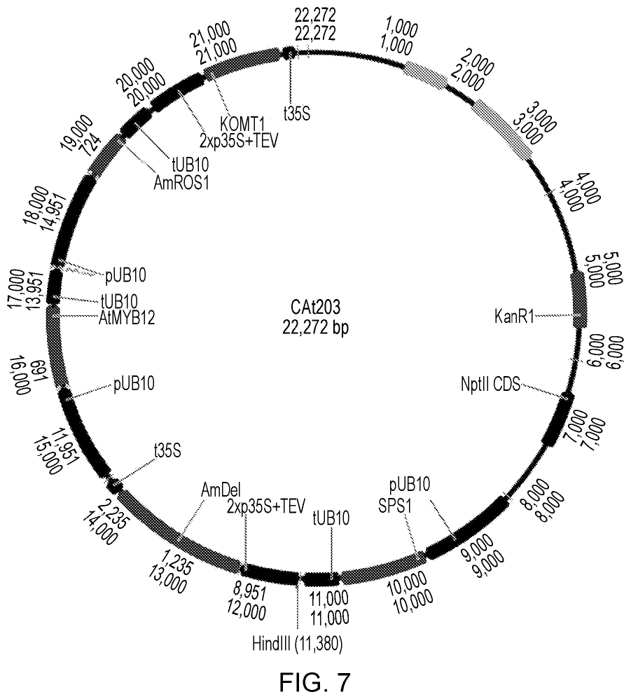

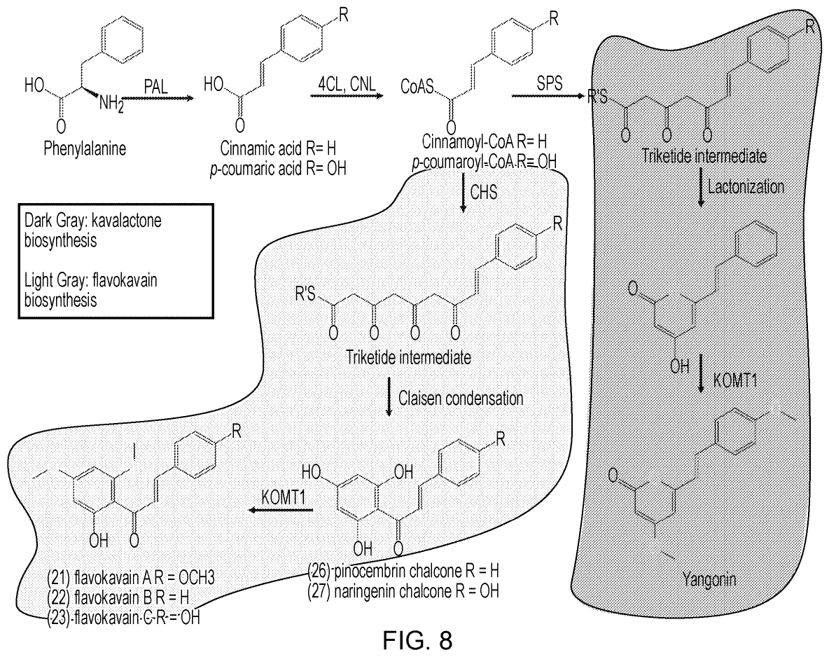

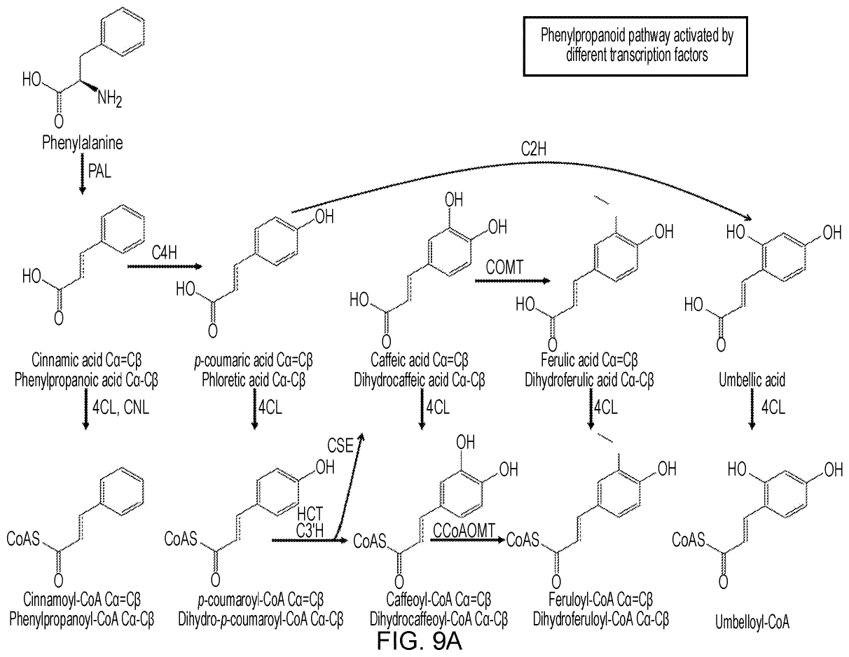

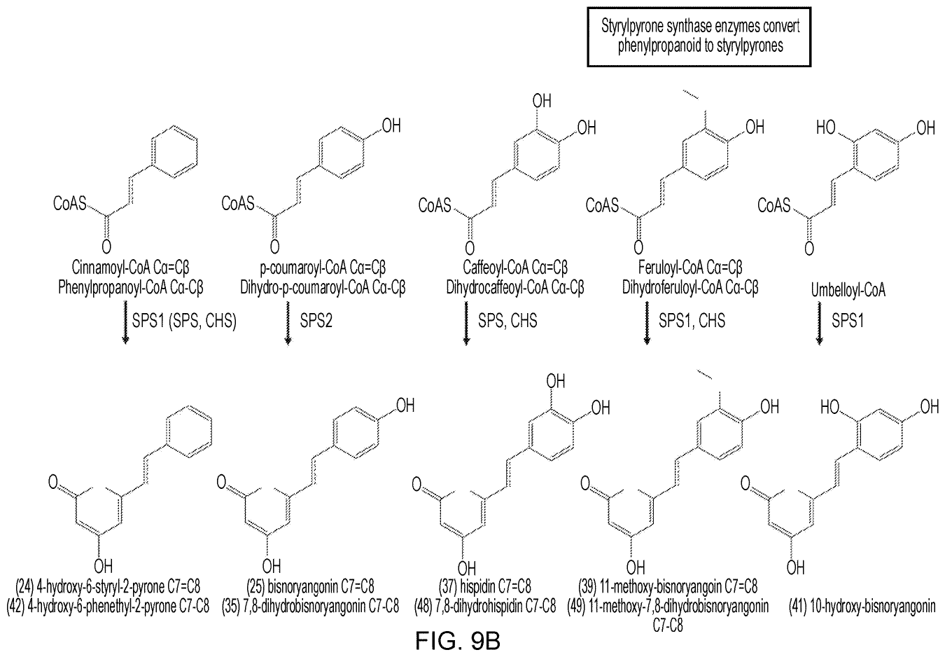

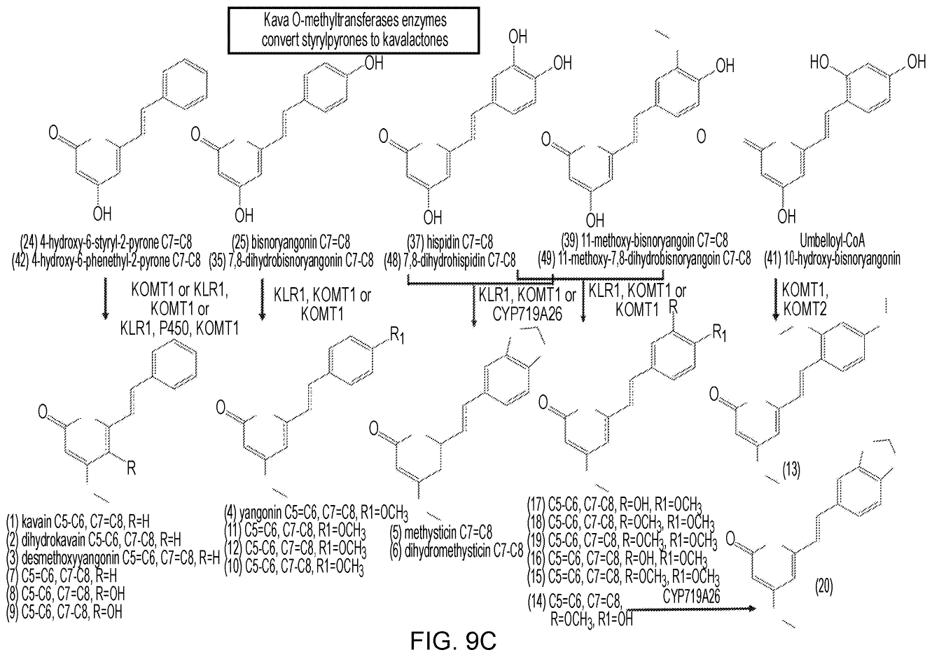

SEQ ID NO. 6 corresponds to the nucleic acid sequence of the Cat203 vector of FIG. 7, which encodes SPS1, KOMT1, AmDEL, AtMYB12 and AmROS1.

### Definitions

This disclosure is not limited by the exemplary methods and materials disclosed herein, and any methods and materials similar or equivalent to those described herein can be used in the practice or testing of embodiments of this disclosure.

The headings provided herein are not limitations of the various aspects or embodiments of this disclosure which can be had by reference to the specification as a whole. Accordingly, the terms defined immediately below are more fully defined by reference to the specification as a whole.

“Kavalactones” are a subset of the “styrylpyrone” family. Therefore, the skilled person would understand that the term “styrylpyrone” encompasses “kavalactones”. Therefore, the term “styrylpyrone” may be replaced by the term “kavalactone” herein.

“Flavokavain” are a subset of the “chalcone” family. Therefore, the skilled person would understand that the term “chalcone” encompasses “flavokavains”. Therefore, the term “chalcone” may be replaced by the term “flavokavain” herein.

Where “enzymes present in the kavalactone or flavokavain pathways” are disclosed, the skilled person will understand that the enzymes may be present in only the kavalactone pathway or only the flavokavain pathway or that the enzymes may be present in both the kavalactone and flavokavain pathways, as some enzymes may be common to both pathways. The skilled person will also understand that the kavalactone and flavokavain pathways may comprise any part of the respective pathways from their common starting point, phenylalanine, through to the resulting kavalactone or flavokavain end point.

The kavalactone pathway may be divided into three subdivisions. The first stage being the phenylpropanoid synthesis pathway; the second stage being the conversion of phenylpropanoids to styrylpyrones; and the third stage being the conversion of the second stage compounds to kavalactones.

The flavokavain pathway may also be divided into three subdivisions. The first stage being the phenylpropanoid synthesis pathway; the second stage being the conversion of phenylpropanoids to chalcones; and the third stage being the conversion of the second stage compounds to flavokavains.

Where “transcription factors present in the flavonoid pathway” are disclosed, the skilled person will understand that the flavonoid pathway may encompass the entire pathway from the phenylalanine starting point through to a flavonoid end product.

The flavonoid pathway may be divided into three subdivisions. The first stage being the phenylpropanoid synthesis pathway; the second stage being early flavonoid biosynthesis; and the third stage being late flavonoid biosynthesis.

It is to be understood that a polypeptide (synonymous with “protein” and/or “enzyme”) may be coded for by more than one nucleotide sequence due to the degeneracy of the genetic code. Therefore, a polynucleotide encoding the vector components of the invention may have at least 70%, 75%, 80%, 85%, 90%, 95%, 96%, 97%, 98% or 99% sequence identity to the disclosed nucleotide sequences (SEQ ID NO: 1, 2, 3, 4, 5, or 6).

Other definitions of terms may appear throughout the specification. Before the exemplary aspects and embodiments are described in more detail, it is important to understand that this disclosure is not limited to particular aspects or embodiments described, which as such may, of course, vary. It is also to be understood that the terminology used herein is for the purpose of describing particular aspects or embodiments only, and is not intended to be limiting, since the scope of the present disclosure will be limited only by the appended claims.

It must be noted that as used herein and in the appended claims, the singular forms “a”, “an”, and “the” include plural referents unless the context clearly dictates otherwise. Thus, for example, reference to “an enzyme” or “a styrylpyrone” or “a chalcone” includes a plurality of such candidate agents and equivalents thereof known to those skilled in the art, and so forth.

### DETAILED DESCRIPTION OF THE INVENTION

Styrylpyrones and chalcones present diverse families of compounds, endogenous to plants and fungi, with a number of bioactive properties. For example, there are over 150 different cultivars of Kava plants, each with varying levels of kavalactones and flavokavains, which are metabolites of styrylpyrones and chalcones. The therapeutic properties of each cultivar differ according to the levels of kavalactones and flavokavains present, thus it would be advantageous to develop methods of controlling the expression of desired compounds.

Furthermore, due to their potential use in food, beverages, pharmaceuticals (including herbal remedies and dietary supplements), cosmetics and personal care products ((Bian et al. Kava as a Clinical Nutrient: Promises and Challenges. Nutrients 2020, 12, 3044), it would be advantageous to develop methods to promote the expression of styrylpyrones or chalcones of interest, such that they can be extracted for further use.

In one aspect, there is provided a composition for promoting the expression of one or more styrylpyrones and/or chalcones in a cell, wherein the composition comprises:
a) one or more first vector components encoding one or more enzymes present in the kavalactone or flavokavain pathways; and
b) one or more second vector components encoding one or more transcription factors present in the flavonoid pathway.

In one embodiment, the one or more first vector components are provided in a first vector and the one or more second vector components are provided in a second vector.

In an alternative embodiment, the one or more first vector components and the one or more second vector components are provided in a single vector.

The skilled person will understand that the number of vectors which comprise the vector components of the composition, will depend on the enzymes present in the kavalactone or flavokavain pathways and the transcription factors present in the flavonoid pathway that they wish to introduce to a cell. The claimed composition provides flexibility to tailor the encoded vector components to the suit the desired promotion of styrylpyrones and/or chalcones. It will be understood that in some embodiments the one or more first vector components may be present in a single vector. In other embodiments the one or more first vector components may be present in more than one vector, such as two, three, four, five, six or more vectors.

It will be understood that in some embodiments the one or more second vector components may be present in a single vector. In other embodiments the one or more second vector components may be present in more than one vector, such as two, three, four, five, six or more vectors.

The number of vectors used for each vector component is independently selected, such that the one or more first vector components may be present in a single vector, whilst the one or more second vector components may be present in more than one vector, and vice versa.

It will also be understood that the invention contemplates all combinations of the vector components disclosed herein. For example, the invention contemplates a first vector comprising one or more first vector components, a second vector comprising one or more second vector components and a third vector comprising one or more first vector components and one or more second vector components.

In one embodiment, the total nucleotide sequence length encoded by the first and/or second vector components is 200 KB or less.

In one embodiment, the total nucleotide sequence length encoded by the first and/or second vector components is 190 KB or less.

In one embodiment, the total nucleotide sequence length encoded by the first and/or second vector components is 180 KB or less.

In one embodiment, the total nucleotide sequence length encoded by the first and/or second vector components is 170 KB or less.

In one embodiment, the total nucleotide sequence length encoded by the first and/or second vector components is 160 KB or less.

In one embodiment, the total nucleotide sequence length encoded by the first and/or second vector components is 150 KB or less.

In one embodiment, the total nucleotide sequence length encoded by the first and/or second vector components is 140 KB, 130 KB, 120 KB, 110 KB, 100 KB, 90 KB, 80 KB, 70 KB, 60 KB, 50 KB, 40 KB, 30 KB, 20 KB, 10 KB or less.

In one embodiment, the total nucleotide sequence length encoded by the first and/or second vector components is 10 KB or more.

In one embodiment, the total nucleotide sequence length encoded by the first and/or second vector components is 20 KB or more.

In one embodiment, the total nucleotide sequence length encoded by the first and/or second vector components is 30 KB or more.

In one embodiment, the total nucleotide sequence length encoded by the first and/or second vector components is 40 KB or more.

In one embodiment, the total nucleotide sequence length encoded by the first and/or second vector components is 50 KB or more.

In one embodiment, the total nucleotide sequence length encoded by the first and/or second vector components is 60 KB, 70 KB, 80 KB, 90 KB, 100 KB, 110 KB, 120 KB, 130 KB, 140 KB, 150 KB, 160 KB, 170 KB, 180 KB, 190 KB, 200 KB or more.

In one embodiment, the total nucleotide sequence length encoded by the first and/or second vector components is between 100 KB and 150 KB.

The total nucleotide sequence length of the first and/or second vector components will depend on whether they are to be inserted independently into two or more vectors, or in combination in a single vector. The skilled person will understand that the total nucleotide sequence length of the vector components is dependent on the vector construct to which they are to be inserted and thus could be greater than 200 KB in certain circumstances.

The skilled person will also understand that the vector construct in which the first vector component and/or second vector component are contained will be selected based on the skilled persons knowledge of suitable vectors for the species of interest. The vector constructs disclosed herein are examples of suitable constructs for algae, moss and plant species. However, other vector constructs may also be appropriate and the skilled person would be aware how to select an appropriate vector construct for their intended species.

Therefore, the skilled person will use their knowledge and the information disclosed herein to determine the total sequence length and the vector construct required for efficient transformation into the species of interest.

In one embodiment, one or more vectors comprising the first vector component and/or second vector component are added to a cell.

In one embodiment, two or more vectors comprising the first vector component and/or second vector component are added to a cell.

In one embodiment, three or more vectors comprising the first vector component and/or second vector component are added to a cell.

In one embodiment, four or more vectors comprising the first vector component and/or second vector component are added to a cell.

In one embodiment, five or more vectors comprising the first vector component and/or second vector component are added to a cell.

In one embodiment, six or more vectors comprising the first vector component and/or second vector component are added to a cell.

In one embodiment, seven or more vectors comprising the first vector component and/or second vector component are added to a cell.

In one embodiment, eight or more vectors comprising the first vector component and/or second vector component are added to a cell.

In one embodiment, nine or more vectors comprising the first vector component and/or second vector component are added to a cell.

In one embodiment, ten or more vectors comprising the first vector component and/or second vector component are added to a cell.

In one embodiment, up to ten vectors comprising the first vector component and/or second vector component are added to a cell.

In a further embodiment, the cell is a plant cell.

In a further embodiment, the plant cell is an algae cell, moss cell, or any cell or cell line derived from the genus Nicotiana.

In yet a further embodiment, the cell or cell line derived from the genus Nicotiana is selected from the group comprising or consisting of Nicotiana tabacum, Nicotiana benthamiana, or BY-2 cells.

In an alternative embodiment, the cell is a yeast cell or a bacterial cell.

The skilled person will understand that the type of vector selected will depend on its intended purpose. The vector may be any suitable type of vector such as a plasmid, cosmid, phage, or viral vector.

In one embodiment, the vector comprising the first vector component and/or second vector component is a plasmid vector.

In a further embodiment, the plasmid vector is a plasmid binary vector.

We have found that the combination of both kavalactone or flavokavain pathway enzymes and transcription factors present in the flavonoid pathway leads to an advantageous expression of various styrylpyrones and/or chalcones in a cell that has been transfected using the composition described herein.

The ability of transcription factors present in the flavonoid pathway to promote the expression of styrylpyrones and/or chalcones is a surprising finding, as the skilled person would expect that the transcription factors present in the flavonoid pathway would drive the expression of flavonoid compounds over styrylpyrones and/or chalcones, thus reducing styrylpyrone and/or chalcone expression in favour of flavonoid expression.

For ease of reference, these and further aspects of the present invention are now discussed under appropriate section headings. However, the teachings under each section are not necessarily limited to each particular section.

### Enzymes Present in the Kavalactone or Flavokavain Pathways

The kavalactone pathway may be divided into three major steps. The first step is the activation of different phenylpropanoid enzymes by different transcription factors. The second step is the conversion of phenylpropanoids to styrylpyrones by styrylpyrone synthase enzymes, such as styrylpyrone biosynthesis 1 (SPS1) and styrylpyrone biosynthesis 2 (SPS2). The third and last step is the conversion of styrylpyrones to kavalactones by Kava O-methyltransferases enzymes.

The flavokavain pathway may also be divided into three steps. The first step is the activation of different phenylpropanoid enzymes by different transcription factors to produce phenylpropanoids. The second step is the conversion of phenylpropanoids to chalcones by chalcone synthase (CHS) and the last step is the conversion of chalcones to flavokavains by kava O-methyltransferase 1 (KOMT1).

Therefore, the kavalactone and flavokavain synthesis pathways both comprise the same early synthesis step encompassed by the phenylpropanoid synthesis pathway (depicted in FIGS. 6 and 7A). From this point, the pathways diverge. The resultant product depends on whether the phenylpropanoid compound is converted by styrylpyrone synthase (to produce styrylpyrones and subsequently kavalactones) or chalcone synthase (to produce chalcones and subsequently flavokavains).

As described herein, the composition for promoting the expression of one or more styrylpyrones and/or chalcones comprises one or more first vector components encoding one or more enzymes present in the kavalactone or flavokavain pathways.

In a further embodiment, the one or more enzymes present in the kavalactone or flavokavain pathways comprise one or more of the group comprising or consisting of phenylalanine ammonia-lyase (PAL), cinnamate 4-hydroxylase (C4H), 4-coumarate:CoA ligase (4CL), hydroxycinnamoyltransferase (HCT), p-coumaroyl shikimate 3′-hydroxylase (C3′H), caffeoyl shikimate esterase (CSE), caffeic acid O-methyltransferase (COMT), caffeoyl-CoA O-methyltransferase (CCoAOMT), coumarate 2-hydroxylase (C2H), cinnamate-CoA ligase (CNL), styrylpyrone biosynthesis 1 (SPS1), styrylpyrone biosynthesis 2 (SPS2), chalcone synthase (CHS), kava O-methyltransferase 1 (KOMT1), kava O-methyltransferase 2 (KOMT2), kavalactone reductase 1 (KLR1), P450, and CYP719A26.

In one embodiment, the first vector component encodes two or more enzymes present in the kavalactone or flavokavain pathways.

In a further embodiment, the two or more enzymes present in the kavalactone or flavokavain pathways comprise two or more of the group comprising or consisting of phenylalanine ammonia-lyase (PAL), cinnamate 4-hydroxylase (C4H), 4-coumarate:CoA ligase (4CL), hydroxycinnamoyltransferase (HCT), p-coumaroyl shikimate 3′-hydroxylase (C3′H), caffeoyl shikimate esterase (CSE), caffeic acid O-methyltransferase (COMT), caffeoyl-CoA O-methyltransferase (CCoAOMT), coumarate 2-hydroxylase (C2H), cinnamate-CoA ligase (CNL), styrylpyrone biosynthesis 1 (SPS1), styrylpyrone biosynthesis 2 (SPS2), chalcone synthase (CHS), kava O-methyltransferase 1 (KOMT1), kava O-methyltransferase 2 (KOMT2), kavalactone reductase 1 (KLR1), P450, and CYP719A26.

In one embodiment, the first vector component encodes three or more enzymes present in the kavalactone or flavokavain pathways.

In a further embodiment, the three or more enzymes present in the kavalactone or flavokavain pathways comprise three or more of the group comprising or consisting of phenylalanine ammonia-lyase (PAL), cinnamate 4-hydroxylase (C4H), 4-coumarate:CoA ligase (4CL), hydroxycinnamoyltransferase (HCT), p-coumaroyl shikimate 3′-hydroxylase (C3′H), caffeoyl shikimate esterase (CSE), caffeic acid O-methyltransferase (COMT), caffeoyl-CoA O-methyltransferase (CCoAOMT), coumarate 2-hydroxylase (C2H), cinnamate-CoA ligase (CNL), styrylpyrone biosynthesis 1 (SPS1), styrylpyrone biosynthesis 2 (SPS2), chalcone synthase (CHS), kava O-methyltransferase 1 (KOMT1), kava O-methyltransferase 2 (KOMT2), kavalactone reductase 1 (KLR1), P450, and CYP719A26.

In one embodiment, the first vector component encodes four or more enzymes present in the kavalactone or flavokavain pathways.

In a further embodiment, the four or more enzymes present in the kavalactone or flavokavain pathways comprise four or more of the group comprising or consisting of phenylalanine ammonia-lyase (PAL), cinnamate 4-hydroxylase (C4H), 4-coumarate:CoA ligase (4CL), hydroxycinnamoyltransferase (HCT), p-coumaroyl shikimate 3′-hydroxylase (C3′H), caffeoyl shikimate esterase (CSE), caffeic acid O-methyltransferase (COMT), caffeoyl-CoA O-methyltransferase (CCoAOMT), coumarate 2-hydroxylase (C2H), cinnamate-CoA ligase (CNL), styrylpyrone biosynthesis 1 (SPS1), styrylpyrone biosynthesis 2 (SPS2), chalcone synthase (CHS), kava O-methyltransferase 1 (KOMT1), kava O-methyltransferase 2 (KOMT2), kavalactone reductase 1 (KLR1), P450, and CYP719A26.

In one embodiment, the first vector component encodes five or more enzymes present in the kavalactone or flavokavain pathways.

In a further embodiment, the five or more enzymes present in the kavalactone or flavokavain pathways comprise five or more of the group comprising or consisting of phenylalanine ammonia-lyase (PAL), cinnamate 4-hydroxylase (C4H), 4-coumarate:CoA ligase (4CL), hydroxycinnamoyltransferase (HCT), p-coumaroyl shikimate 3′-hydroxylase (C3′H), caffeoyl shikimate esterase (CSE), caffeic acid O-methyltransferase (COMT), caffeoyl-CoA O-methyltransferase (CCoAOMT), coumarate 2-hydroxylase (C2H), cinnamate-CoA ligase (CNL), styrylpyrone biosynthesis 1 (SPS1), styrylpyrone biosynthesis 2 (SPS2), chalcone synthase (CHS), kava O-methyltransferase 1 (KOMT1), kava O-methyltransferase 2 (KOMT2), kavalactone reductase 1 (KLR1), P450, and CYP719A26.

In one embodiment, the first vector component encodes six or more enzymes present in the kavalactone or flavokavain pathways.

In a further embodiment, the six or more enzymes present in the kavalactone or flavokavain pathways comprise six or more of the group comprising or consisting of phenylalanine ammonia-lyase (PAL), cinnamate 4-hydroxylase (C4H), 4-coumarate:CoA ligase (4CL), hydroxycinnamoyltransferase (HCT), p-coumaroyl shikimate 3′-hydroxylase (C3′H), caffeoyl shikimate esterase (CSE), caffeic acid O-methyltransferase (COMT), caffeoyl-CoA O-methyltransferase (CCoAOMT), coumarate 2-hydroxylase (C2H), cinnamate-CoA ligase (CNL), styrylpyrone biosynthesis 1 (SPS1), styrylpyrone biosynthesis 2 (SPS2), chalcone synthase (CHS), kava O-methyltransferase 1 (KOMT1), kava O-methyltransferase 2 (KOMT2), kavalactone reductase 1 (KLR1), P450, and CYP719A26.

In one embodiment, the first vector component encodes seven or more enzymes present in the kavalactone or flavokavain pathways.

In a further embodiment, the seven or more enzymes present in the kavalactone or flavokavain pathways comprise seven or more of the group comprising or consisting of phenylalanine ammonia-lyase (PAL), cinnamate 4-hydroxylase (C4H), 4-coumarate:CoA ligase (4CL), hydroxycinnamoyltransferase (HCT), p-coumaroyl shikimate 3′-hydroxylase (C3′H), caffeoyl shikimate esterase (CSE), caffeic acid O-methyltransferase (COMT), caffeoyl-CoA O-methyltransferase (CCoAOMT), coumarate 2-hydroxylase (C2H), cinnamate-CoA ligase (CNL), styrylpyrone biosynthesis 1 (SPS1), styrylpyrone biosynthesis 2 (SPS2), chalcone synthase (CHS), kava O-methyltransferase 1 (KOMT1), kava O-methyltransferase 2 (KOMT2), kavalactone reductase 1 (KLR1), P450, and CYP719A26.

In one embodiment, the first vector component encodes eight or more enzymes present in the kavalactone or flavokavain pathways.

In a further embodiment, the eight or more enzymes present in the kavalactone or flavokavain pathways comprise eight or more of the group comprising or consisting of phenylalanine ammonia-lyase (PAL), cinnamate 4-hydroxylase (C4H), 4-coumarate:CoA ligase (4CL), hydroxycinnamoyltransferase (HCT), p-coumaroyl shikimate 3′-hydroxylase (C3′H), caffeoyl shikimate esterase (CSE), caffeic acid O-methyltransferase (COMT), caffeoyl-CoA O-methyltransferase (CCoAOMT), coumarate 2-hydroxylase (C2H), cinnamate-CoA ligase (CNL), styrylpyrone biosynthesis 1 (SPS1), styrylpyrone biosynthesis 2 (SPS2), chalcone synthase (CHS), kava O-methyltransferase 1 (KOMT1), kava O-methyltransferase 2 (KOMT2), kavalactone reductase 1 (KLR1), P450, and CYP719A26.

In one embodiment, the first vector component encodes nine or more enzymes present in the kavalactone or flavokavain pathways.

In a further embodiment, the nine or more enzymes present in the kavalactone or flavokavain pathways comprise nine or more of the group comprising or consisting of phenylalanine ammonia-lyase (PAL), cinnamate 4-hydroxylase (C4H), 4-coumarate:CoA ligase (4CL), hydroxycinnamoyltransferase (HCT), p-coumaroyl shikimate 3′-hydroxylase (C3′H), caffeoyl shikimate esterase (CSE), caffeic acid O-methyltransferase (COMT), caffeoyl-CoA O-methyltransferase (CCoAOMT), coumarate 2-hydroxylase (C2H), cinnamate-CoA ligase (CNL), styrylpyrone biosynthesis 1 (SPS1), styrylpyrone biosynthesis 2 (SPS2), chalcone synthase (CHS), kava O-methyltransferase 1 (KOMT1), kava O-methyltransferase 2 (KOMT2), kavalactone reductase 1 (KLR1), P450, and CYP719A26.

In one embodiment, the first vector component encodes ten or more enzymes present in the kavalactone or flavokavain pathways.

In a further embodiment, the ten or more enzymes present in the kavalactone or flavokavain pathways comprise ten or more of the group comprising or consisting of phenylalanine ammonia-lyase (PAL), cinnamate 4-hydroxylase (C4H), 4-coumarate:CoA ligase (4CL), hydroxycinnamoyltransferase (HCT), p-coumaroyl shikimate 3′-hydroxylase (C3′H), caffeoyl shikimate esterase (CSE), caffeic acid O-methyltransferase (COMT), caffeoyl-CoA O-methyltransferase (CCoAOMT), coumarate 2-hydroxylase (C2H), cinnamate-CoA ligase (CNL), styrylpyrone biosynthesis 1 (SPS1), styrylpyrone biosynthesis 2 (SPS2), chalcone synthase (CHS), kava O-methyltransferase 1 (KOMT1), kava O-methyltransferase 2 (KOMT2), kavalactone reductase 1 (KLR1), P450, and CYP719A26.

In one embodiment, the first vector component encodes eleven or more enzymes present in the kavalactone or flavokavain pathways.

In a further embodiment, the eleven or more enzymes present in the kavalactone or flavokavain pathways comprise eleven or more of the group comprising or consisting of phenylalanine ammonia-lyase (PAL), cinnamate 4-hydroxylase (C4H), 4-coumarate:CoA ligase (4CL), hydroxycinnamoyltransferase (HCT), p-coumaroyl shikimate 3′-hydroxylase (C3′H), caffeoyl shikimate esterase (CSE), caffeic acid O-methyltransferase (COMT), caffeoyl-CoA O-methyltransferase (CCoAOMT), coumarate 2-hydroxylase (C2H), cinnamate-CoA ligase (CNL), styrylpyrone biosynthesis 1 (SPS1), styrylpyrone biosynthesis 2 (SPS2), chalcone synthase (CHS), kava O-methyltransferase 1 (KOMT1), kava O-methyltransferase 2 (KOMT2), kavalactone reductase 1 (KLR1), P450, and CYP719A26.

In one embodiment, the one or more second vector components encode 12, 13, 14, 15, 16, 17, 18, 19, 20, 21, 22, 23, 24, or 25 or more enzymes present in the kavalactone or flavokavain pathways.

In a further embodiment, the 12, 13, 14, 15, 16, 17, 18, 19, 20, 21, 22, 23, 24, or 25 or more enzymes present in the kavalactone or flavokavain pathways comprise 12, 13, 14, 15, 16, 17, 18, 19, 20, 21, 22, 23, 24, or 25 or more of the group comprising phenylalanine ammonia-lyase (PAL), cinnamate 4-hydroxylase (C4H), 4-coumarate:CoA ligase (4CL), hydroxycinnamoyltransferase (HCT), p-coumaroyl shikimate 3′-hydroxylase (C3′H), caffeoyl shikimate esterase (CSE), caffeic acid O-methyltransferase (COMT), caffeoyl-CoA O-methyltransferase (CCoAOMT), coumarate 2-hydroxylase (C2H), cinnamate-CoA ligase (CNL), styrylpyrone biosynthesis 1 (SPS1), styrylpyrone biosynthesis 2 (SPS2), chalcone synthase (CHS), kava O-methyltransferase 1 (KOMT1), kava O-methyltransferase 2 (KOMT2), kavalactone reductase 1 (KLR1), P450, and CYP719A26. PAL, C4H, 4CL, HCT, C3′H, CSE, COMT, CCOAOMT, C2H, and CNL are all enzymes associated with phenylpropanoid metabolism, which represents the first stage of the kavalactone and flavokavain pathways. Increased expression of one or more of these enzymes leads to over expression of phenylpropanoids, which are then catalysed to form styrylpyrones and chalcones and subsequently kavalactones and flavokavains respectively. These enzymes may therefore be referred to herein as ‘enzymes present in the phenylpropanoid pathway’.

In one embodiment, the one or more enzymes present in the kavalactone or flavokavain pathways are selected from the group comprising enzymes present in the phenylpropanoid pathway.

The skilled person will be aware that the maximum number of enzymes encoded by the first vector component will be dependent on the constraints of the vector construct chosen. E.g. the total sequence length that the vector construct can support.

In one embodiment, the one or more enzymes present in the kavalactone or flavokavain pathways are one or more enzymes present in the kavalactone pathway.

In a further embodiment, the one or more enzymes present in the kavalactone pathway comprise or consist of styrylpyrone biosynthesis (SPS) and kava O-methyltransferase 1 (KOMT1).

In yet a further embodiment SPS is SPS1 or SPS2.

In yet another further embodiment SPS is SPS1.

In yet another further embodiment SPS is SPS2.

In one embodiment, the one or more enzymes present in the kavalactone or flavokavain pathways are one or more enzymes present in the flavokavain pathway.

In one embodiment, the one or more enzymes present in the flavokavain pathway comprise or consist of kava O-methyltransferase 1 (KOMT1).

Styrylpyrone biosynthesis (SPS) is a polyketide synthase (PKS), that catalyses the reaction which forms the styrylpyrone backbone. Kava O-methyltransferase 1 (KOMT1) is capable of methylating the styrylpyrone backbone or, specifically the —OH groups at the C4, C11 or C12 positions. The skilled person will understand that not all the styrylpyrones generated by SPS will be catalysed by KOMT1 to form kavalactones. Furthermore, phenylpropanoid precursor compounds may be converted by endogenous or encoded CHS, instead of SPS, and enter into the flavokavain pathway. Chalcones generated as part of the flavokavain pathway may be catalysed by KOMT1 to form flavokavains. Thus, the combination of these enzymes advantageously leads to the expression of various styrylpyrones, their kavalactone metabolites, chalcones, and flavokavains.

Described below are non-limiting examples of combinations of enzymes present in the kavalactone or flavokavain pathways encoded by the vector component, which may be chosen by the skilled person to promote the expression of desired styrylpyrones and/or chalcones in cells.

In one embodiment, the one or more enzymes present in the kavalactone or flavokavain pathways is SPS1.

In one embodiment, the one or more enzymes present in the kavalactone or flavokavain pathways are SPS1 and the enzymes present in the phenylpropanoid pathway.

In one embodiment, the one or more enzymes present in the kavalactone or flavokavain pathways is SPS2.

In one embodiment, the one or more enzymes present in the kavalactone or flavokavain pathways are SPS2 and the enzymes present in the phenylpropanoid pathway.

In one embodiment, the one or more enzymes present in the kavalactone or flavokavain pathways is CHS.

In one embodiment, the one or more enzymes present in the kavalactone or flavokavain pathways are CHS and the enzymes present in the phenylpropanoid pathway.

In one embodiment, the one or more enzymes present in the kavalactone or flavokavain pathways are one or more of SPS1, SPS2, CHS, and the enzymes present in the phenylpropanoid pathway.

In one embodiment, the one or more enzymes present in the kavalactone or flavokavain pathways is KOMT1.

In one embodiment, the one or more enzymes present in the kavalactone or flavokavain pathways are KOMT and the enzymes present in the phenylpropanoid pathway.

In one embodiment, the one or more enzymes present in the kavalactone or flavokavain pathways are KOMT1 and SPS1.

In one embodiment, the one or more enzymes present in the kavalactone or flavokavain pathways are KOMT1, SPS1, and the enzymes present in the phenylpropanoid pathway.

In one embodiment, the one or more enzymes present in the kavalactone or flavokavain pathways are KOMT1 and SPS2.

In one embodiment, the one or more enzymes present in the kavalactone or flavokavain pathways are KOMT1, SPS2, and the enzymes present in the phenylpropanoid pathway.

In one embodiment, the one or more enzymes present in the kavalactone or flavokavain pathways are KOMT1 and CHS.

In one embodiment, the one or more enzymes present in the kavalactone or flavokavain pathways are KOMT1, CHS, and the enzymes present in the phenylpropanoid pathway.

In one embodiment, the one or more enzymes present in the kavalactone or flavokavain pathways are KOMT1 and one or more of SPS1, SPS2, CHS, and the enzymes present in the phenylpropanoid pathway.

In one embodiment, the one or more enzymes present in the kavalactone or flavokavain pathways are KOMT1 and KOMT2.

In one embodiment, the one or more enzymes present in the kavalactone or flavokavain pathways are KOMT1, KOMT2, and the enzymes present in the phenylpropanoid pathway

In one embodiment, the one or more enzymes present in the kavalactone or flavokavain pathways are KOMT1, KOMT2, and SPS1.

In one embodiment, the one or more enzymes present in the kavalactone or flavokavain pathways are KOMT1, KOMT2, SPS1, and the enzymes present in the phenylpropanoid pathway.

In one embodiment, the one or more enzymes present in the kavalactone or flavokavain pathways are KOMT1, KOMT2, and SPS2.

In one embodiment, the one or more enzymes present in the kavalactone or flavokavain pathways are KOMT1, KOMT2, SPS2, and the enzymes present in the phenylpropanoid pathway.

In one embodiment, the one or more enzymes present in the kavalactone or flavokavain pathways are KOMT1, KOMT2, and CHS.

In one embodiment, the one or more enzymes present in the kavalactone or flavokavain pathways are KOMT1, KOMT2, CHS, and the enzymes present in the phenylpropanoid pathway.

In one embodiment, the one or more enzymes present in the kavalactone or flavokavain pathways are KOMT1, KOMT2, and one or more of SPS1, SPS2, CHS, and the enzymes present in the phenylpropanoid pathway.

In one embodiment, the one or more enzymes present in the kavalactone or flavokavain pathways are KOMT1 and CYP719A26.

In one embodiment, the one or more enzymes present in the kavalactone or flavokavain pathways are KOMT1, CYP719A26, and the enzymes present in the phenylpropanoid pathway.

In one embodiment, the one or more enzymes present in the kavalactone or flavokavain pathways are KOMT1, CYP719A26, and SPS1.

In one embodiment, the one or more enzymes present in the kavalactone or flavokavain pathways are KOMT1, CYP719A26, SPS1, and the enzymes present in the phenylpropanoid pathway.

In one embodiment, the one or more enzymes present in the kavalactone or flavokavain pathways are KOMT1, CYP719A26, and SPS2.

In one embodiment, the one or more enzymes present in the kavalactone or flavokavain pathways are KOMT1, CYP719A26, SPS2, and the enzymes present in the phenylpropanoid pathway.

In one embodiment, the one or more enzymes present in the kavalactone or flavokavain pathways are KOMT1, CYP719A26, and CHS.

In one embodiment, the one or more enzymes present in the kavalactone or flavokavain pathways are KOMT1, CYP719A26, CHS, and the enzymes present in the phenylpropanoid pathway.

In one embodiment, the one or more enzymes present in the kavalactone or flavokavain pathways are KOMT1, CYP719A26, and one or more of SPS1, SPS2, CHS, and the enzymes present in the phenylpropanoid pathway.

In one embodiment, the one or more enzymes present in the kavalactone or flavokavain pathways are KOMT1, CYP719A26, KLR1, and SPS1.

In one embodiment, the one or more enzymes present in the kavalactone or flavokavain pathways are KOMT1, CYP719A26, KLR1, SPS1, and the enzymes present in the phenylpropanoid pathway.

In one embodiment, the one or more enzymes present in the kavalactone or flavokavain pathways are KOMT1, CYP719A26, KLR1, and SPS2.

In one embodiment, the one or more enzymes present in the kavalactone or flavokavain pathways are KOMT1, CYP719A26, KLR1, SPS2, and the enzymes present in the phenylpropanoid pathway.

In one embodiment, the one or more enzymes present in the kavalactone or flavokavain pathways are KOMT1, CYP719A26, KLR1, and CHS.

In one embodiment, the one or more enzymes present in the kavalactone or flavokavain pathways are KOMT1, CYP719A26, KLR1, CHS, and the enzymes present in the phenylpropanoid pathway.

In one embodiment, the one or more enzymes present in the kavalactone or flavokavain pathways are KOMT1, CYP719A26, KLR1, and one or more of SPS1, SPS2, CHS, and the enzymes present in the phenylpropanoid pathway.

In one embodiment, the one or more enzymes present in the kavalactone or flavokavain pathways are KOMT1, and KLR1.

In one embodiment, the one or more enzymes present in the kavalactone or flavokavain pathways are KOMT1, KLR1, and the enzymes present in the phenylpropanoid pathway.

In one embodiment, the one or more enzymes present in the kavalactone or flavokavain pathways are KOMT1, KLR1, and SPS1.

In one embodiment, the one or more enzymes present in the kavalactone or flavokavain pathways are KOMT1, KLR1, SPS1, and the enzymes present in the phenylpropanoid pathway.

In one embodiment, the one or more enzymes present in the kavalactone or flavokavain pathways are KOMT1, KLR1, and SPS2.

In one embodiment, the one or more enzymes present in the kavalactone or flavokavain pathways are KOMT1, KLR1, SPS2, and the enzymes present in the phenylpropanoid pathway.

In one embodiment, the one or more enzymes present in the kavalactone or flavokavain pathways are KOMT1, KLR1, and CHS.

In one embodiment, the one or more enzymes present in the kavalactone or flavokavain pathways are KOMT1, KLR1, CHS, and the enzymes present in the phenylpropanoid pathway.

In one embodiment, the one or more enzymes present in the kavalactone or flavokavain pathways are KOMT1, KLR1, and one or more of SPS1, SPS2, CHS, and the enzymes present in the phenylpropanoid pathway.

In one embodiment, the one or more enzymes present in the kavalactone or flavokavain pathways are KOMT1, KLR1, and P450.

In one embodiment, the one or more enzymes present in the kavalactone or flavokavain pathways are KOMT1, KLR1, P450, and the enzymes present in the phenylpropanoid pathway.

In one embodiment, the one or more enzymes present in the kavalactone or flavokavain pathways are KOMT1, KLR1, P450 and SPS1.

In one embodiment, the one or more enzymes present in the kavalactone or flavokavain pathways are KOMT1, KLR1, P450, SPS1, and the enzymes present in the phenylpropanoid pathway.

In one embodiment, the one or more enzymes present in the kavalactone or flavokavain pathways are KOMT1, KLR1, P450 and SPS2.

In one embodiment, the one or more enzymes present in the kavalactone or flavokavain pathways are KOMT1, KLR1, P450, SPS2, and the enzymes present in the phenylpropanoid pathway.

In one embodiment, the one or more enzymes present in the kavalactone or flavokavain pathways are KOMT1, KLR1, P450 and CHS.

In one embodiment, the one or more enzymes present in the kavalactone or flavokavain pathways are KOMT1, KLR1, P450, CHS, and the enzymes present in the phenylpropanoid pathway.

In one embodiment, the one or more enzymes present in the kavalactone or flavokavain pathways are KOMT1, KLR1, P450 and one or more of SPS1, SPS2, CHS, and the enzymes present in the phenylpropanoid pathway.

The skilled person understands that enzymes present in the kavalactone or flavokavain pathways may be isolated from a variety of different plant species. Thus, a plurality of different isoforms may exist for each enzyme described herein.

In selecting the combination of enzymes present in the kavalactone or flavokavain pathways the skilled person will use their knowledge of the kavalactone and flavokavain pathways to select appropriate enzymes to reach the desired styrylpyrone and/or chalcone.

### Transcription Factors Present in the Flavonoid Pathway

Flavonoids are plant secondary polyphenolic metabolites, which can be divided into six major subgroups according to their molecular structures. They are endogenously expressed in higher plants; however, the early biosynthetic steps of the pathway have also been found in the bryophytes Physcomitrella patens and Marchantia polymorpha, suggesting that flavonoids may have originally evolved as chemical messengers (Li. Trancriptional control of flavonoid biosynthesis, Plant Signaling & Behavior, 2014, 9, e27522). Flavonoid biosynthesis is principally modulated at the transcriptional level, thus there are a variety of transcription factors associated with the various biosynthetic steps.

As described herein, the composition for promoting the expression of one or more styrylpyrones and/or chalcones comprises a second vector component encoding one or more transcription factors present in the flavonoid pathway.

In one embodiment, the one or more transcription factors present in the flavonoid pathway are selected from the group comprising or consisting of myeloblastosis (MYB) type transcription factors and/or basic helix-loop-helix (bHLH) type transcription factors.

In a further embodiment, the one or more transcription factors present in the flavonoid pathway are independently derived from a plant species.

The skilled person will understand that the transcription factors listed above and below may be derived from different plant species. Therefore, the listed transcription factors are intended to encompass those derived from any species including, but not necessarily limited to the prefixes At, Vv, Ib, Gh, Gm, Md, Gt, Ms, Co, Ej, Mg, Pc, Ppy, Pb, Par, Pi, Pdm, Pav, Pcr, Pcf, Pd, Ppr, Ps, Fa, Fv, Rh, Ri, Hv, Bo, Mr, Eg, Es, In, Dk, Mt, Lh, Lj, Zm, Pp, Cs, Og, Py, Pt, Ptr, Pto, St, Ta, Nt, and Le.

The skilled person will also understand that different plant species have their own transcription factors present in the flavonoid pathway. Therefore, the lists of transcription factors present in the flavonoid pathway provided above are non-limiting and the skilled person would select the most appropriate transcription factors based on the plant species of interest.

In yet a further embodiment, the plant species is selected from the group comprising or consisting of At (Arabidopsis thaliana), Am (Antirrhinum majus), Nt (Nicotiana tabacum), Mt (Medicago truncatula), Vv (Vitis vinifera), or Cs (Camellia sinensis).

In one embodiment, the second vector component encodes two or more transcription factors present in the flavonoid pathway.

In one embodiment, the two or more transcription factors present in the flavonoid pathway are selected from the group comprising or consisting of myeloblastosis (MYB) type transcription factors and/or basic helix-loop-helix (bHLH) type transcription factors. In a further embodiment, the two or more transcription factors present in the flavonoid pathway are independently derived from a plant species.

In yet a further embodiment, the plant species is selected from the group comprising or consisting of At (Arabidopsis thaliana), Am (Antirrhinum majus), Nt (Nicotiana tabacum), Mt (Medicago truncatula), Vv (Vitis vinifera), or Cs (Camellia sinensis).

In one embodiment, the second vector component encodes three or more transcription factors present in the flavonoid pathway.

In one embodiment, the three or more transcription factors present in the flavonoid pathway are selected from the group comprising or consisting of myeloblastosis (MYB) type transcription factors and/or basic helix-loop-helix (bHLH) type transcription factors.

In a further embodiment, the three or more transcription factors present in the flavonoid pathway are independently derived from a plant species.

In yet a further embodiment, the plant species is selected from the group comprising or consisting of At (Arabidopsis thaliana), Am (Antirrhinum majus), Nt (Nicotiana tabacum), Mt (Medicago truncatula), Vv (Vitis vinifera), or Cs (Camellia sinensis).

In one embodiment, the second vector component encodes four or more transcription factors present in the flavonoid pathway.

In one embodiment, the four or more transcription factors present in the flavonoid pathway are selected from the group comprising or consisting of myeloblastosis (MYB) type transcription factors and/or basic helix-loop-helix (bHLH) type transcription factors.

In a further embodiment, the four or more transcription factors present in the flavonoid pathway are independently derived from a plant species.

In yet a further embodiment, the plant species is selected from the group comprising or consisting of At (Arabidopsis thaliana), Am (Antirrhinum majus), Nt (Nicotiana tabacum), Mt (Medicago truncatula), Vv (Vitis vinifera), or Cs (Camellia sinensis).

In one embodiment, the second vector component encodes five or more transcription factors present in the flavonoid pathway.

In one embodiment, the five or more transcription factors present in the flavonoid pathway are selected from the group comprising or consisting of myeloblastosis (MYB) type transcription factors and/or basic helix-loop-helix (bHLH) type transcription factors.

In a further embodiment, the five or more transcription factors present in the flavonoid pathway are independently derived from a plant species.

In yet a further embodiment, the plant species is selected from the group comprising or consisting of At (Arabidopsis thaliana), Am (Antirrhinum majus), Nt (Nicotiana tabacum), Mt (Medicago truncatula), Vv (Vitis vinifera), or Cs (Camellia sinensis).

In one embodiment, the second vector component encodes six or more transcription factors present in the flavonoid pathway.

In one embodiment, the six or more transcription factors present in the flavonoid pathway are selected from the group comprising or consisting of myeloblastosis (MYB) type transcription factors and/or basic helix-loop-helix (bHLH) type transcription factors.

In a further embodiment, the six or more transcription factors present in the flavonoid pathway are independently derived from a plant species.

In yet a further embodiment, the plant species is selected from the group comprising or consisting of At (Arabidopsis thaliana), Am (Antirrhinum majus), Nt (Nicotiana tabacum), Mt (Medicago truncatula), Vv (Vitis vinifera), or Cs (Camellia sinensis).

In one embodiment, the second vector component encodes 7, 8, 9, 10, 11, 12, 13, 14, 15, 16, 17, 18, 19, 20, 21, 22, 23, 24, 25, or more transcription factors present in the flavonoid pathway.

In one embodiment, the 7, 8, 9, 10, 11, 12, 13, 14, 15, 16, 17, 18, 19, 20, 21, 22, 23, 24, 25, or more transcription factors present in the flavonoid pathway are selected from the group comprising or consisting of myeloblastosis (MYB) type transcription factors and/or basic helix-loop-helix (bHLH) type transcription factors.

In a further embodiment, the 7, 8, 9, 10, 11, 12, 13, 14, 15, 16, 17, 18, 19, 20, 21, 22, 23, 24, 25, or more transcription factors present in the flavonoid pathway are independently derived from a plant species.

In yet a further embodiment, the plant species is selected from the group comprising or consisting of At (Arabidopsis thaliana), Am (Antirrhinum majus), Nt (Nicotiana tabacum), Mt (Medicago truncatula), Vv (Vitis vinifera), or Cs (Camellia sinensis).

The skilled person will be aware that the maximum number of transcription factors encoded by the second vector component will be dependent on the constraints of the vector construct chosen. E.g. the total sequence length that the vector construct can support.

In embodiments where each transcription factor is independently derived from a plant species, it should be noted that each transcription factor need not necessarily be derived from the same plant species.

MYB proteins are transcription factors found in all eukaryotes. However, whilst only a few unique MYBs are encoded in unikonts (the phylogenetic group comprising animals and fungi), there are up to ~200 unique MYB genes found in the plant lineage. Of the MYB proteins present in plants, R2R3 MYB transcription factors are the most widely encoded, with many plant species encoding >100 unique R2R3 MYB.

In one embodiment, the MYB type transcription factor is a member of the R2R3 MYB transcription factor family.

In one embodiment, the MYB type transcription factor is one or more of MYB0, MYB1, MYB10, MYB100, MYB101, MYB102, MYB103, MYB104, MYB105, MYB106, MYB107, MYB108, MYB109, MYB11, MYB110, MYB111, MYB112, MYB113, MYB114, MYB115, MYB116, MYB117, MYB118, MYB119, MYB12, MYB120, MYB121, MYB122, MYB123 (TT2), MYB13, MYB14, MYB15, MYB16, MYB17, MYB18, MYB19, MYB2, MYB20, MYB21, MYB22, MYB23, MYB24, MYB25, MYB26, MYB27, MYB28, MYB29, MYB3, MYB30, MYB31, MYB32, MYB33, MYB34, MYB35, MYB36, MYB37, MYB38, MYB39, MYB3R, MYB3R1, MYB3R2, MYB3R3, MYB3R4, MYB3R5, MYB4, MYB40, MYB41, MYB42, MYB43, MYB44, MYB45, MYB46, MYB47, MYB48, MYB49, MYB5, MYB50, MYB51, MYB52, MYB53, MYB54, MYB55, MYB56, MYB57, MYB58, MYB59, MYB6, MYB60, MYB61, MYB62, MYB63, MYB64, MYB65, MYB66, MYB67, MYB68, MYB69, MYB7, MYB70, MYB71, MYB72, MYB73, MYB74, MYB75 (PAP1 or PAPA1), MYB76, MYB77, MYB78, MYB79, MYB8, MYB80, MYB81, MYB82, MYB83, MYB84, MYB85, MYB86, MYB87, MYB88, MYB89, MYB9, MYB90 (PAP2 or PAPA2), MYB91, MYB92, MYB93, MYB94, MYB95, MYB96, MYB97, MYB98, MYB99, ANT1, AN2, A, MYB1a, P, C1, ROSEA1 (ROS1), ROSEA2 (ROS2), VENOSA, MBF1, MYBA, MYB, MYB110a, PURPLE (PR), GMYB10, VIMYBA1-1, VIMYBA1-2, VIMYBA1-3, VIMYBA2, MYBA1, MYBA2, MYBF1, MYBPA1, MYBPA2, MYB5a, MYB5b, MYBP3, MYBP4, LEGUME ANTHOCYANIN PRODUCTION1 (LAP1), PAR, MYB12-Lat, TT2a, TT2b, TT2c, PL, PL-BLOTCHED1 (PL-BH), P1, MYB-IF35, RUBY, DP, L, PHZ, MYB134, MYB216, AN1, MTF1, Y1, MYB176, MYB-G20-1, MYB12B2.

In one embodiment, the MYB type transcription factor is one or more of MYB1, MYB3, MYB10, MYB110a, MYBA, MYB11, MYB12, MYB14, MYB15, MYB46, MYB58, MYB61, MYB63, MYB75 (PAP1), MYB83, MYB85, MYB90 (PAP2), MYB111, MYB113, MYB114, MYB123 (TT2), MYB2, PURPLE (PR), GMYB10, VIMYBA1-1, VIMYBA1-2, VIMYBA1-3, VIMYBA2, MYBA1, MYBA2, MYBF1, MYBPA1, MYBPA2, MYB5a, MYB5b, MYBP3, MYBP4, MYB4, LEGUME ANTHOCYANIN PRODUCTION1 (LAP1), PAR, MYB6, MYB12-Lat, TT2a, TT2b, TT2c, C1, PL, PL-BLOTCHED1 (PL-BH), P1, MYB-IF35, RUBY, AN2, DP, L, PHZ, MYB20, MYB134, MYB216, AN1, MTF1, ROS1, ROS2, VENOSA, Y1, MYB176, MYB-G20-1, MYB12B2, MYB9, A, ANT1.

In one embodiment, the MYB type transcription factor is MYB12.

In one embodiment, the MYB type transcription factor is AtMYB12.

In one embodiment, the MYB type transcription factor is ROS1.

In one embodiment, the MYB type transcription factor is AmROS1.

In one embodiment, the MYB type transcription factor is at least one of MYB12 and ROS1.

In one embodiment, the MYB type transcription factor is at least one of AtMYB12, and AmROS1.

In one embodiment, the MYB type transcription factor is one or more of MYB11, MYB111, and MYB12.

In one embodiment, the MYB type transcription factor is one or more of AtMYB11, AtMYB111, and AtMYB12.

In one embodiment, the MYB type transcription factor is one or more of MYB75, MYBF1, MYBPA, and MYBA.

In one embodiment, the MYB type transcription factor is one or more of AtMYB75, AtMYBF1, AtMYBPA, and AtMYBA.

bHLH type transcription factors are so named due to their highly conserved alkaline/helix-loop-helix domain. Originally identified in animals, they have also been found in plants and are involved in diverse functions including plant growth and development, via the functions of DNA binding and dimerization

In one embodiment, the bHLH type transcription factor is one or more of DEL, TT8, GL3, EGL3, and MYC1.

In a further embodiment, DEL, TT8, GL3, EGL3, and MYC1 are AmDEL, AtTT8, AtGL3, AtEGL3, and AtMYC1.

In one embodiment, the bHLH type transcription factor is DEL.

In one embodiment, the bHLH type transcription factor is AmDEL.

In one embodiment, the one or more transcription factors present in the flavonoid pathway are selected from the group comprising or consisting of MYB12, DEL, and ROS1.

In a further embodiment, MYB12, DEL, and ROS1 are AtMYB12, AmDEL, and AmROS1.

In one embodiment, the one or more transcription factors present in the flavonoid pathway are selected from the group comprising or consisting of AN2, TT8, and MYB12.

In a further embodiment, AN2, TT8, and MYB12 are NtAN2, AtTT8, and AtMYB12.

The skilled person will understand that different plant species have their own transcription factors present in the flavonoid pathway. Therefore, the lists of transcription factors present in the flavonoid pathway provided above are non-limiting and the skilled person would select the most appropriate transcription factors based on the plant species of interest.

The ability of transcription factors present in the flavonoid pathway to promote the expression of styrylpyrones and chalcones is a surprising finding. The skilled person would have expected that the expression of flavonoids would have been promoted over non-flavonoid compounds. For example, we have found that for other pathways, such as curcuminoids, which share the phenylpropanoid pathway with styrylpyrones and chalcones as a first stage, the addition of transcription factors present in the flavonoid pathway reduces the expression of the target compound. Thus, it would not have been obvious to the skilled person to attempt to promote expression of styrylpyrones and chalcones with transcription factors present in the flavonoid pathway.

Styrylpyrones and Chalcones Obtainable from the Composition

As has already been disclosed, styrylpyrones are a group of phenylpropanoid natural products found in plants and fungi. Styrylpyrones and metabolites of styrylpyrones, such as kavalactones, may exert neuroprotective properties, in particular significant anxiolytic, analgesic, or neuroprotective effects. Therefore, styrylpyrones present a diverse range of commercially attractive compounds.

As described herein, the composition promotes the expression of one or more styrylpyrones and/or chalcones.

In one embodiment, the one or more styrylpyrones and/or chalcones are one or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2,trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, katsumadain C, pinocembrin chalcone, naringenin chalcone, flavokavain A, flavokavain B, and flavokavain C, or derivatives thereof.

In one embodiment, the composition promotes the expression of one or more styrylpyrones.

In one embodiment, the one or more styrylpyrones are one or more kavalactones.

In a further embodiment, the one or more kavalactones comprise one or more plant-derived kavalactones.

In one embodiment, the one or more styrylpyrones comprise one or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangoinin, 10-methoxyangoinin, 11-hydroxyangoinin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2, trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, and katsumadain C, or derivatives thereof.

In one embodiment, the one or more styrylpyrones comprise one or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivaties thereof.

In one embodiment, the one or more styrylpyrones comprise one or more of the group comprising or consisting of 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the one or more styrylpyrones comprise one or more of the group comprising or consisting of kavain, 7,8-dihydrokavain, desmethoxyyangonin, yangonin, methysticin, and 7,8-dihydromethysticin, or derivatives thereof.

In one embodiment, the one or more styrylpyrones is yangonin, or a derivative thereof.

In one embodiment, the one or more styrylpyrones is kavain, or a derivative thereof.

In one embodiment, the one or more styrylpyrones is 7,8-dihydrokavain, or a derivative thereof.

In one embodiment, the one or more styrylpyrones is desmethoxyyangonin, or a derivative thereof.

In one embodiment, the one or more styrylpyrones is methysticin, or a derivative thereof.

In one embodiment, the one or more styrylpyrones is 7,8-dihydromethysticin, or a derivative thereof.

In one embodiment, the composition promotes the expression of one or more chalcones, or a derivative thereof.

In one embodiment, the one or more chalcones are one or more flavokavains, or a derivative thereof.

In one embodiment, the one or more chalcones are one or more of the group comprising or consisting of pinocembrin chalcone, naringenin chalcone, flavokavain A, flavokavain B, and flavokavain C, or derivatives thereof.

In one embodiment, the one or more chalcones is pinocembrin chalcone, or a derivative thereof.

In one embodiment, the one or more chalcones is naringenin chalcone, or a derivative thereof.

In one embodiment, the one or more chalcones is flavokavain A, or a derivative thereof.

In one embodiment, the one or more chalcones is flavokavain B, or a derivative thereof.

In one embodiment, the one or more chalcones is flavokavain C, or a derivative thereof.

In one embodiment, the composition promotes the expression of two or more styrylpyrones and/or chalcones.

In one embodiment, the two or more styrylpyrones and/or chalcones are two or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2, trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, katsumadain C, pinocembrin chalcone, naringenin chalcone, flavokavain A, flavokavain B, and flavokavain C, or derivatives thereof.

In one embodiment, the composition promotes the expression of two or more styrylpyrones.

In one embodiment, the two or more styrylpyrones are two or more kavalactones.

In a further embodiment, the two or more kavalactones comprise two or more plant-derived kavalactones.

In one embodiment, the two or more styrylpyrones comprise two or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2, trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5′S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, and katsumadain C, or derivatives thereof.

In one embodiment, the two or more styrylpyrones comprise two or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the two or more styrylpyrones comprise two or more of the group comprising or consisting of 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the two or more styrylpyrones comprise two or more of the group comprising or consisting of kavain, 7,8-dihydrokavain, desmethoxyyangonin, yangonin, methysticin, and 7,8-dihydromethysticin, or derivatives thereof.

In one embodiment, the two or more styrylpyrones are yangonin, or a derivative thereof and at least one other styrylpyrone.

In one embodiment, the two or more styrylpyrones are kavain, or a derivative thereof and at least one other styrylpyrone.

In one embodiment, the two or more styrylpyrones are 7,8-dihydrokavain, or a derivative thereof and at least one other styrylpyrone.

In one embodiment, the two or more styrylpyrones are desmethoxyyangonin, or a derivative thereof and at least one other styrylpyrone.

In one embodiment, the two or more styrylpyrones are methysticin, or a derivative thereof and at least one other styrylpyrone.

In one embodiment, the two or more styrylpyrones are 7,8-dihydromethysticin, or a derivative thereof and at least one other styrylpyrone.

In one embodiment, the composition promotes the expression of two or more chalcones.

In one embodiment, the two or more chalcones are two or more flavokavains.

In one embodiment, the two or more chalcones are two or more of the group comprising or consisting of pinocembrin chalcone, naringenin chalcone, flavokavain A, flavokavain B, and flavokavain C, or derivatives thereof.

In one embodiment, the two or more chalcones are pinocembrin chalcone, or a derivative thereof and at least one other chalcone.

In one embodiment, the two or more chalcones are naringenin chalcone, or a derivative thereof and at least one other chalcone.

In one embodiment, the two or more chalcones are flavokavain A, or a derivative thereof and at least one other chalcone.

In one embodiment, the two or more chalcones are flavokavain B, or a derivative thereof and at least one other chalcone.

In one embodiment, the two or more chalcones are flavokavain C, or a derivative thereof and at least one other chalcone.

In one embodiment, the composition promotes the expression of three or more styrylpyrones and/or chalcones.

In one embodiment, the three or more styrylpyrones and/or chalcones are three or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2, trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, katsumadain C, pinocembrin chalcone, naringenin chalcone, flavokavain A, flavokavain B, and flavokavain C, or derivatives thereof.

In one embodiment, the composition promotes the expression of three or more styrylpyrones.

In one embodiment, the three or more styrylpyrones are three or more kavalactones.

In a further embodiment, the three or more kavalactones comprise three or more plant-derived kavalactones.

In one embodiment, the three or more styrylpyrones comprise three or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2, trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, and katsumadain C, or derivatives thereof.

In one embodiment, the three or more styrylpyrones comprise three or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the three or more styrylpyrones comprise three or more of the group comprising or consisting of 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the three or more styrylpyrones comprise three or more of the group comprising or consisting of kavain, 7,8-dihydrokavain, desmethoxyyangonin, yangonin, methysticin, and 7,8-dihydromethysticin, or derivatives thereof.

In one embodiment, the three or more styrylpyrones are yangonin, or a derivative thereof and at least two other styrylpyrones.

In one embodiment, the three or more styrylpyrones are kavain, or a derivative thereof and at least two other styrylpyrones.

In one embodiment, the three or more styrylpyrones are 7,8-dihydrokavain, or a derivative thereof and at least two other styrylpyrones.

In one embodiment, the three or more styrylpyrones are desmethoxyyangonin, or a derivative thereof and at least two other styrylpyrones.

In one embodiment, the three or more styrylpyrones are methysticin, or a derivative thereof and at least two other styrylpyrones.

In one embodiment, the three or more styrylpyrones are 7,8-dihydromethysticin, or a derivative thereof and at least two other styrylpyrones.

In one embodiment, the composition promotes the expression of three or more chalcones.

In one embodiment, the three or more chalcones are three or more flavokavains.

In one embodiment, the three or more chalcones are three or more of the group comprising or consisting of pinocembrin chalcone, naringenin chalcone, flavokavain A, flavokavain B, and flavokavain C, or derivatives thereof.

In one embodiment, the three or more chalcones are flavokavain A, flavokavain B, and flavokavain C, or derivatives thereof.

In one embodiment, the three or more chalcones are pinocembrin chalcone, or a derivative thereof and at least two other chalcones.

In one embodiment, the three or more chalcones are naringenin chalcone, or a derivative thereof and at least two other chalcones.

In one embodiment, the three or more chalcones are flavokavain A, or a derivative thereof and at least two other chalcones.

In one embodiment, the three or more chalcones are flavokavain B, or a derivative thereof and at least two other chalcones.

In one embodiment, the three or more chalcones are flavokavain C, or a derivative thereof and at least two other chalcones.

In one embodiment, the composition promotes the expression of four or more styrylpyrones and/or chalcones.

In one embodiment, the four or more styrylpyrones and/or chalcones are four or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2, trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, katsumadain C, pinocembrin chalcone, naringenin chalcone, flavokavain A, flavokavain B, and flavokavain C, or derivatives thereof.

In one embodiment, the composition promotes the expression of four or more styrylpyrones.

In one embodiment, the four or more styrylpyrones are four or more kavalactones.

In a further embodiment, the four or more kavalactones comprise four or more plant-derived kavalactones.

In one embodiment, the four or more styrylpyrones comprise four or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2,trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, and katsumadain C, or derivatives thereof.

In one embodiment, the four or more styrylpyrones comprise four or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the four or more styrylpyrones comprise four or more of the group comprising or consisting of 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the four or more styrylpyrones comprise four or more of the group comprising or consisting of kavain, 7,8-dihydrokavain, desmethoxyyangonin, yangonin, methysticin, and 7,8-dihydromethysticin, or derivatives thereof.

In one embodiment, the four or more styrylpyrones are yangonin, or a derivative thereof and at least three other styrylpyrones.

In one embodiment, the four or more styrylpyrones are kavain, or a derivative thereof and at least three other styrylpyrones.

In one embodiment, the four or more styrylpyrones are 7,8-dihydrokavain, or a derivative thereof and at least three other styrylpyrones.

In one embodiment, the four or more styrylpyrones are desmethoxyyangonin, or a derivative thereof and at least three other styrylpyrones.

In one embodiment, the four or more styrylpyrones are methysticin, or a derivative thereof and at least three other styrylpyrones.

In one embodiment, the four or more styrylpyrones are 7,8-dihydromethysticin, or a derivative thereof and at least three other styrylpyrones.

In one embodiment, the composition promotes the expression of four or more chalcones.

In one embodiment, the four or more chalcones are four or more of the group comprising or consisting of pinocembrin chalcone, naringenin chalcone, flavokavain A, flavokavain B, and flavokavain C, or derivatives thereof.

In one embodiment, the four or more chalcones are flavokavain A, flavokavain B, flavokavain C, or derivatives thereof and at least one other chalcone.

In one embodiment, the four or more chalcones are pinocembrin chalcone, or a derivative thereof and at least three other chalcones.

In one embodiment, the four or more chalcones are naringenin chalcone, or a derivative thereof and at least three other chalcones.

In one embodiment, the four or more chalcones are flavokavain A, or a derivative thereof and at least three other chalcones.

In one embodiment, the four or more chalcones are flavokavain B, or a derivative thereof and at least three other chalcones.

In one embodiment, the four or more chalcones are flavokavain C, or a derivative thereof and at least three other chalcones.

In one embodiment, the composition promotes the expression of five or more styrylpyrones and/or chalcones.

In one embodiment, the five or more styrylpyrones and/or chalcones are five or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2,trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, katsumadain C, pinocembrin chalcone, naringenin chalcone, flavokavain A, flavokavain B, and flavokavain C, or derivatives thereof.

In one embodiment, the composition promotes the expression of five or more styrylpyrones.

In one embodiment, the five or more styrylpyrones are five or more kavalactones.

In a further embodiment, the five or more kavalactones comprise five or more plant-derived kavalactones.

In one embodiment, the five or more styrylpyrones comprise five or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2,trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, and katsumadain C, or derivatives thereof.

In one embodiment, the five or more styrylpyrones comprise five or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the five or more styrylpyrones comprise five or more of the group comprising or consisting of 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the five or more styrylpyrones comprise five or more of the group comprising or consisting of kavain, 7,8-dihydrokavain, desmethoxyyangonin, yangonin, methysticin, and 7,8-dihydromethysticin, or derivatives thereof.

In one embodiment, the five or more styrylpyrones are yangonin, or a derivative thereof and at least four other styrylpyrones.

In one embodiment, the five or more styrylpyrones are kavain, or a derivative thereof and at least four other styrylpyrones.

In one embodiment, the five or more styrylpyrones are 7,8-dihydrokavain, or a derivative thereof and at least four other styrylpyrones.

In one embodiment, the five or more styrylpyrones are desmethoxyyangonin, or a derivative thereof and at least four other styrylpyrones.

In one embodiment, the five or more styrylpyrones are methysticin, or a derivative thereof and at least four other styrylpyrones.

In one embodiment, the five or more styrylpyrones are 7,8-dihydromethysticin, or a derivative thereof and at least four other styrylpyrones.

In one embodiment, the composition promotes the expression of five or more chalcones.

In one embodiment, the five or more chalcones are pinocembrin chalcone, naringenin chalcone, flavokavain A, flavokavain B, and flavokavain C, or derivatives thereof.

In one embodiment, the five or more chalcones are flavokavain A, flavokavain B, flavokavain C, and at least two other chalcones, or derivatives thereof.

In one embodiment, the five or more chalcones are pinocembrin chalcone, or a derivative thereof and at least four other chalcones.

In one embodiment, the five or more chalcones are naringenin chalcone, or a derivative thereof and at least four other chalcones.

In one embodiment, the five or more chalcones are flavokavain A, or a derivative thereof and at least four other chalcones.

In one embodiment, the five or more chalcones are flavokavain B, or a derivative thereof and at least four other chalcones.

In one embodiment, the five or more chalcones are flavokavain C, or a derivative thereof and at least four other chalcones.

In one embodiment, the composition promotes the expression of 6, 7, 8, 9, 10, 11, 12, 13, 14, 15 or more styrylpyrones and/or chalcones.

In one embodiment, the 6, 7, 8, 9, 10, 11, 12, 13, 14, 15 or more styrylpyrones and/or chalcones are 6, 7, 8, 9, 10, 11, 12, 13, 14, 15 or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2, trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, katsumadain C, pinocembrin chalcone, naringenin chalcone, flavokavain A, flavokavain B, and flavokavain C, or derivatives thereof.

In one embodiment, the composition promotes the expression of 6, 7, 8, 9, 10, 11, 12, 13, 14, 15 or more styrylpyrones.

In one embodiment, the 6, 7, 8, 9, 10, 11, 12, 13, 14, 15 or more styrylpyrones are 6, 7, 8, 9, 10, 11, 12, 13, 14, 15 or more kavalactones.

In a further embodiment, the 6, 7, 8, 9, 10, 11, 12, 13, 14, 15 or more kavalactones comprise 6, 7, 8, 9, 10, 11, 12, 13, 14, 15 or more plant-derived kavalactones.

In one embodiment, the 6, 7, 8, 9, 10, 11, 12, 13, 14, 15 or more styrylpyrones comprise 6, 7, 8, 9, 10, 11, 12, 13, 14, 15 or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2,trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, and katsumadain C, or derivatives thereof.

In one embodiment, the 6, 7, 8, 9, 10, 11, 12, 13, 14, 15 or more styrylpyrones comprise 6, 7, 8, 9, 10, 11, 12, 13, 14, 15 or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the 6, 7, 8, 9, 10, 11, 12, 13, 14, 15 or more styrylpyrones comprise 6, 7, 8, 9, 10, 11, 12, 13, 14, 15 or more of the group comprising or consisting of 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the 6, 7, 8, 9, 10, 11, 12, 13, 14, 15 or more styrylpyrones comprise kavain, 7,8-dihydrokavain, desmethoxyyangonin, yangonin, methysticin, 7,8-dihydromethysticin, or derivatives thereof and at least 0, 1, 2, 3, 4, 5, 6, 7, 8, 9 other styrylpyrones.

In one embodiment, the 6, 7, 8, 9, 10, 11, 12, 13, 14, 15 or more styrylpyrones are yangonin, or a derivative thereof and at least 5, 6, 7, 8, 9, 10, 11, 12, 13, 14 other styrylpyrones.

In one embodiment, the 6, 7, 8, 9, 10, 11, 12, 13, 14, 15 or more styrylpyrones are kavain, or a derivative thereof and at least 5, 6, 7, 8, 9, 10, 11, 12, 13, 14 other styrylpyrones.

In one embodiment, the 6, 7, 8, 9, 10, 11, 12, 13, 14, 15 or more styrylpyrones are 7,8-dihydrokavain, or a derivative thereof and at least 5, 6, 7, 8, 9, 10, 11, 12, 13, 14 other styrylpyrones.

In one embodiment, the 6, 7, 8, 9, 10, 11, 12, 13, 14, 15 or more styrylpyrones are desmethoxyyangonin, or a derivative thereof and at least 5, 6, 7, 8, 9, 10, 11, 12, 13, 14 other styrylpyrones.

In one embodiment, the 6, 7, 8, 9, 10, 11, 12, 13, 14, 15 or more styrylpyrones are methysticin, or a derivative thereof and at least 5, 6, 7, 8, 9, 10, 11, 12, 13, 14 other styrylpyrones.

In one embodiment, the 6, 7, 8, 9, 10, 11, 12, 13, 14, 15 or more styrylpyrones are 7,8-dihydromethysticin, or a derivative thereof and at least 5, 6, 7, 8, 9, 10, 11, 12, 13, 14 other styrylpyrones.

The skilled person will understand that due to the nature of catalysis reactions, not all intermediate metabolites will be catalysed to form the next metabolite. The selected combinations of enzymes present in the kavalactone or flavokavain pathways and transcription factors present in the flavonoid pathway will promote the expression of particular styrylpyrones, kavalactones, chalcones and/or flavokavains. However, intermediate metabolites of the pathway may also be present. Therefore, a plurality of styrylpyrone, kavalactone, chalcone and/or flavokavain metabolites may be expressed in the cell transfected with the vector components described herein.

In certain embodiments, the styrylpyrone or chalcone obtained and the combination of enzymes present the kavalactone or flavokavain pathways and transcription factors present in the flavonoid pathway to obtain the styrylpyrone or chalcone are as recited in Table 1.

Table 1 provides non-limiting examples of combinations of enzymes present in the kavalactone or flavokavain pathways and transcription factors present in the flavonoid pathway, encoded by the vector components as described herein, used to obtain styrylpyrones, kavalactones, chalcones and/or flavokavains. The enzyme required for conversion of phenylpropanoids to chalcones, CHS, may be endogenous to the transfected cell. Therefore, it is not listed in this table, but is not excluded from being included in the first vector component.

| | Enzymes present in | Example combination |
|---|---|---|
| | the kavalactone or | of transcription factors |
| | flavokavain | present in the flavonoid |
| Styrylpyrone or chalcone obtained | pathways | pathway: |
| 4-hydroxy-6-styryl-2-pyrone C7═C8 | SPS1 or SPS2 | MYB12, DEL, ROS1 |
| bisnoryangonin C7═C8 | SPS2 | MYB12, DEL, ROS1 |
| 7,8-dihydrobisnoryangonin C7—C8 | SPS2 | MYB12, DEL, ROS1 |
| hispidin C7═C8 | SPS2 | MYB12, DEL, ROS1 |
| 11-methoxy-bisnoryangonin C7═C8 | SPS1 | MYB12, DEL, ROS1 |
| 10-hydroxy-bisnoryangonin | SPS1 | MYB12, DEL, ROS1 |
| 4-hydroxy-6-phenethyl-2-pyrone C7—C8 | SPS1 or SPS2 | MYB12, DEL, ROS1 |
| 7,8-dihydrohispidin C7—C8 | SPS2 | MYB12, DEL, ROS1 |
| 11-methoxy-7,8- | SPS1 | MYB12, DEL, ROS1 |
| dihydrobisnoryangoin C7—C8 | | |
| 11-hydroxy-12-methoxydihydrokavain | KLR1, KOMT1; and | MYB12, DEL, ROS1 |
| | SPS1 or SPS2 | |
| 7,8-dihydrokavain | KLR1, KOMT1; and | MYB12, DEL, ROS1 |
| | SPS1 or SPS2 | |
| 7,8-dihydromethysticin | KLR1, KOMT1, | MYB12, DEL, ROS1 |
| | CYP719A26, and | |
| | SPS2 | |
| 5,6,7,8-tetrahydroyangonin | KLR1, KOMT1, and | MYB12, DEL, ROS1 |
| | SPS2 | |
| 11,12-dimethoxy-5,6-dihydrokavain | KLR1, KOMT1; and | MYB12, DEL, ROS1 |
| | SPS1 or SPS2 | |
| 7,8-dihydro-5-hydroxykavain | KLR1, P450, KOMT1; | MYB12, DEL, ROS1 |
| | and SPS1 or SPS2, | |
| kavain | KLR1, KOMT1, and | MYB12, DEL, ROS1 |
| | SPS1 or SPS2 | |
| methysticin | KLR1, KOMT1, | MYB12, DEL, ROS1 |
| | CYP719A26, and | |
| | SPS2 | |
| 5,6-dihydroyangonin | KLR1, KOMT1, and | MYB12, DEL, ROS1 |
| | SPS2 | |
| 5-hydroxykavain | KLR1, P450, KOMT1; | MYB12, DEL, ROS1 |
| | and SPS1 or SPS2 | |
| 5,6-dehydromethysticin | KOMT1, | MYB12, DEL, ROS1 |
| | CYP719A26; and | |
| | SPS1 or SPS2 | |
| 7,8-dihydroyangonin | KOMT1; and SPS1 or | MYB12, DEL, ROS1 |
| | SPS2 | |
| desmethoxyyangonin | KOMT1; and SPS1 or | MYB12, DEL, ROS1 |
| | SPS2 | |
| yangonin | KOMT1; and SPS1 or | MYB12, DEL, ROS1 |
| | SPS2 | |
| 11-methoxyangonin | KOMT1 and SPS2 | MYB12, DEL, ROS1 |
| 10-methoxyangonin | KOMT1, KOMT2, and | MYB12, DEL, ROS1 |
| | SPS1 | |
| 11-hydroxyangonin | KOMT1 and SPS2 | MYB12, DEL, ROS1 |
| 11-methoxy-12- | KOMT1; and SPS1 or | MYB12, DEL, ROS1 |
| hydroxydehydrokavain | SPS2 | |
| 11-methoxy-5,6-dihydroyangonin | KLR1, KOMT1; and | MYB12, DEL, ROS1 |
| | SPS1 or SPS2 | |
| 7,8-dihydro-5,6-dehydrokavain | KOMT1; and SPS1 or | MYB12, DEL, ROS1 |
| | SPS2 | |
| 4-methoxy-6-(11,12- | KOMT1; and SPS1 or | MYB12, DEL, ROS1 |
| methylenedioxydihydrostyryl)-2- | SPS2 | |
| pyrone | | |
| 4-methoxy-6-(11,12-methylenedioxy- | KOMT1; and SPS1 or | MYB12, DEL, ROS1 |
| 14-methoxy-dihydrostyryl)-2-pyrone | SPS2 | |
| 4-methoxy-6(11,-12-methylenedioxy- | KOMT1; and SPS1 or | MYB12, DEL, ROS1 |
| 10,14-dimethoxydihydrostyryl)-2- | SPS2 | |
| pyrone | | |
| 4-methoxy-6-(11,12-methylenedioxy- | KOMT1, KOMT2, and | MYB12, DEL, ROS1 |
| 14-methoxystyryl)-2-pyrone | SPS1 | |
| 4-methoxy-6-(11,12- | KOMT1, KOMT2, and | MYB12, DEL, ROS1 |
| methylenedioxystyryl)-2- | SPS1 | |
| pyrone | | |
| 4-methoxy-6-(11,12-dimethylstyryl)-2- | KOMT1, KOMT2, and | MYB12, DEL, ROS1 |
| pyrone | SPS1 | |
| pinocembrin chalcone | CHS* | MYB12, DEL, ROS1 |
| naringenin chalcone | CHS* | MYB12, DEL, ROS1 |
| Flavokavain A | KOMT1 | MYB12, DEL, ROS1 |
| Flavokavain B | KOMT1 | MYB12, DEL, ROS1 |
| Flavokavain C | KOMT1 | MYB12, DEL, ROS1 |

*may be endogenous to the cell and thus not required to be encoded by the first vector component.

As is also exemplified in table 1, it is clear that there are combinations of enzymes, present in the kavalactone or flavokavain pathways, common to multiple styrylpyrones and/or chalcones. Thus, a selected combination of enzymes present in the kavalactone or flavokavain pathways, encoded by the vector components, has the potential to promote the expression of a plurality of styrylpyrones and/or chalcones.

### Plant Products

As described herein, in one aspect there is provided a plant, plant part or plant cell transformed with the vector components according to any one of the composition embodiments described herein.

In one embodiment, the plant, plant part or plant cell is algae, moss or any plant, plant part or plant cell derived from the genus Nicotiana.

In a further embodiment, the plant, plant part or plant cell derived from the genus Nicotiana is selected from the group comprising or consisting of Nicotiana tabacum, Nicotiana benthamiana, or BY-2 cells.

### Use

As described herein, in one aspect there is provided use of the composition according to any one of the composition embodiments described herein for promoting the expression of one or more styrylpyrones and/or chalcones in algae, moss, or any plant, plant part, or plant cells.

In one embodiment, the plant cells are a cell line derived from plants.

In one embodiment, the plant, plant part, or plant cells are derived from the genus Nicotiana.

In yet a further embodiment, the plant, plant part or plant cells derived from the genus Nicotiana are selected from the group comprising or consisting of Nicotiana tabacum, Nicotiana benthamiana, or BY-2 cells.

In an alternative embodiment, the expression of one or more styrylpyrones and/or chalcones is promoted in yeast cells and/or bacterial cells.

In one embodiment, the composition promotes the expression of one or more styrylpyrones and/or chalcones.

In one embodiment, the one or more styrylpyrones and/or chalcones are one or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2, trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, katsumadain C, pinocembrin chalcone, naringenin chalcone, flavokavain A, flavokavain B, and flavokavain C, or derivatives thereof.

In one embodiment, the composition promotes the expression of one or more styrylpyrones.

In one embodiment, the one or more styrylpyrones comprise one or more kavalactones.

In a further embodiment, the one or more kavalactones comprise one or more plant-derived kavalactones.

In one embodiment, the one or more styrylpyrones comprise one or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2,trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, and katsumadain C, or derivatives thereof.

In one embodiment, the one or more styrylpyrones comprise one or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the one or more styrylpyrones comprise one or more of the group comprising or consisting of 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the one or more styrylpyrones comprise one or more of the group comprising or consisting of kavain, 7,8-dihydrokavain, desmethoxyyangonin, yangonin, methysticin, and 7,8-dihydromethysticin, or derivatives thereof.

In one embodiment, the one or more styrylpyrones is yangonin, or a derivative thereof.

In one embodiment, the one or more styrylpyrones is kavain, or a derivative thereof.

In one embodiment, the one or more styrylpyrones is 7,8-dihydrokavain, or a derivative thereof.

In one embodiment, the one or more styrylpyrones is desmethoxyyangonin, or a derivative thereof.

In one embodiment, the one or more styrylpyrones is methysticin, or a derivative thereof.

In one embodiment, the one or more styrylpyrones is 7,8-dihydromethysticin, or a derivative thereof.

In one embodiment, the composition promotes the expression of one or more chalcones.

In one embodiment, the one or more chalcones are one or more flavokavains.

In one embodiment, the one or more chalcones are one or more of the group comprising or consisting of pinocembrin chalcone, naringenin chalcone, flavokavain A, flavokavain B, and flavokavain C, or derivatives thereof.

In one embodiment, the one or more chalcones is pinocembrin chalcone, or a derivative thereof.

In one embodiment, the one or more chalcones is naringenin chalcone, or a derivative thereof.

In one embodiment, the one or more chalcones is flavokavain A, or a derivative thereof.

In one embodiment, the one or more chalcones is flavokavain B, or a derivative thereof. In one embodiment, the one or more chalcones is flavokavain C, or a derivative thereof.

In one embodiment, the composition promotes the expression of two or more styrylpyrones and/or chalcones.

In one embodiment, the two or more styrylpyrones and/or chalcones are two or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2,trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, katsumadain C, pinocembrin chalcone, naringenin chalcone, flavokavain A, flavokavain B, and flavokavain C, or derivatives thereof.

In one embodiment, the composition promotes the expression of two or more styrylpyrones.

In one embodiment, the two or more styrylpyrones are two or more kavalactones.

In a further embodiment, the two or more kavalactones comprise two or more plant-derived kavalactones.

In one embodiment, the two or more styrylpyrones comprise two or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2, trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, and katsumadain C, or derivatives thereof.

In one embodiment, the two or more styrylpyrones comprise two or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the two or more styrylpyrones comprise two or more of the group comprising or consisting of 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the two or more styrylpyrones comprise two or more of the group comprising or consisting of kavain, 7,8-dihydrokavain, desmethoxyyangonin, yangonin, methysticin, and 7,8-dihydromethysticin, or derivatives thereof.

In one embodiment, the two or more styrylpyrones are yangonin, or a derivative thereof and at least one other styrylpyrone.

In one embodiment, the two or more styrylpyrones are kavain, or a derivative thereof and at least one other styrylpyrone.

In one embodiment, the two or more styrylpyrones are 7,8-dihydrokavain, or a derivative thereof and at least one other styrylpyrone.

In one embodiment, the two or more styrylpyrones are desmethoxyyangonin, or a derivative thereof and at least one other styrylpyrone.

In one embodiment, the two or more styrylpyrones are methysticin, or a derivative thereof and at least one other styrylpyrone.

In one embodiment, the two or more styrylpyrones are 7,8-dihydromethysticin, or a derivative thereof and at least one other styrylpyrone.

In one embodiment, the composition promotes the expression of two or more chalcones.

In one embodiment, the two or more chalcones are two or more flavokavains.

In one embodiment, the two or more chalcones are two or more of the group comprising or consisting of pinocembrin chalcone, naringenin chalcone, flavokavain A, flavokavain B, and flavokavain C, or derivatives thereof.

In one embodiment, the two or more chalcones are pinocembrin chalcone, or a derivative thereof and at least one other chalcone.

In one embodiment, the two or more chalcones are naringenin chalcone, or a derivative thereof and at least one other chalcone.

In one embodiment, the two or more chalcones are flavokavain A, or a derivative thereof and at least one other chalcone.

In one embodiment, the two or more chalcones are flavokavain B, or a derivative thereof and at least one other chalcone.

In one embodiment, the two or more chalcones are flavokavain C, or a derivative thereof and at least one other chalcone.

In one embodiment, the composition promotes the expression of three or more styrylpyrones and/or chalcones.

In one embodiment, the three or more styrylpyrones and/or chalcones are three or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2, trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, katsumadain C, pinocembrin chalcone, naringenin chalcone, flavokavain A, flavokavain B, and flavokavain C, or derivatives thereof.

In one embodiment, the composition promotes the expression of three or more styrylpyrones.

In one embodiment, the three or more styrylpyrones are three or more kavalactones.

In a further embodiment, the three or more kavalactones comprise three or more plant-derived kavalactones.

In one embodiment, the three or more styrylpyrones comprise three or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2, trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, and katsumadain C, or derivatives thereof.

In one embodiment, the three or more styrylpyrones comprise three or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the three or more styrylpyrones comprise three or more of the group comprising or consisting of 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the three or more styrylpyrones comprise three or more of the group comprising or consisting of kavain, 7,8-dihydrokavain, desmethoxyyangonin, yangonin, methysticin, and 7,8-dihydromethysticin, or deriviatives thereof.

In one embodiment, the three or more styrylpyrones are yangonin, or a derivative thereof and at least two other styrylpyrones.

In one embodiment, the three or more styrylpyrones are kavain, or a derivative thereof and at least two other styrylpyrones.

In one embodiment, the three or more styrylpyrones are 7,8-dihydrokavain, or a derivative thereof and at least two other styrylpyrones.

In one embodiment, the three or more styrylpyrones are desmethoxyyangonin, or a derivative thereof and at least two other styrylpyrones.

In one embodiment, the three or more styrylpyrones are methysticin, or a derivative thereof and at least two other styrylpyrones.

In one embodiment, the three or more styrylpyrones are 7,8-dihydromethysticin, or a derivative thereof and at least two other styrylpyrones.

In one embodiment, the composition promotes the expression of three or more chalcones.

In one embodiment, the three or more chalcones are three or more of the group comprising or consisting of pinocembrin chalcone, naringenin chalcone, flavokavain A, flavokavain B, and flavokavain C, or derivatives thereof.

In one embodiment, the three or more chalcones are flavokavain A, flavokavain B, and flavokavain C, or derivatives thereof.

In one embodiment, the three or more chalcones are pinocembrin chalcone, or a derivative thereof and at least two other chalcones.

In one embodiment, the three or more chalcones are naringenin chalcone, or a derivative thereof and at least two other chalcones.

In one embodiment, the three or more chalcones are flavokavain A, or a derivative thereof and at least two other chalcones.

In one embodiment, the three or more chalcones are flavokavain B, or a derivative thereof and at least two other chalcones.

In one embodiment, the three or more chalcones are flavokavain C, or a derivative thereof and at least two other chalcones.

In one embodiment, the composition promotes the expression of four or more styrylpyrones and/or chalcones.

In one embodiment, the four or more styrylpyrones and/or chalcones are four or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2,trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, katsumadain C, pinocembrin chalcone, naringenin chalcone, flavokavain A, flavokavain B, and flavokavain C, or derivatives thereof.

In one embodiment, the composition promotes the expression of four or more styrylpyrones.

In one embodiment, the four or more styrylpyrones are four or more kavalactones.

In a further embodiment, the four or more kavalactones comprise four or more plant-derived kavalactones.

In one embodiment, the four or more styrylpyrones comprise four or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2, trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, and katsumadain C, or derivatives thereof.

In one embodiment, the four or more styrylpyrones comprise four or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the four or more styrylpyrones comprise four or more of the group comprising or consisting of 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the four or more styrylpyrones comprise four or more of the group comprising or consisting of kavain, 7,8-dihydrokavain, desmethoxyyangonin, yangonin, methysticin, and 7,8-dihydromethysticin, or derivatives thereof.

In one embodiment, the four or more styrylpyrones are yangonin, or a derivative thereof and at least three other styrylpyrones.

In one embodiment, the four or more styrylpyrones are kavain, or a derivative thereof and at least three other styrylpyrones.

In one embodiment, the four or more styrylpyrones are 7,8-dihydrokavain, or a derivative thereof and at least three other styrylpyrones.

In one embodiment, the four or more styrylpyrones are desmethoxyyangonin, or a derivative thereof and at least three other styrylpyrones.

In one embodiment, the four or more styrylpyrones are methysticin, or a derivative thereof and at least three other styrylpyrones.

In one embodiment, the four or more styrylpyrones are 7,8-dihydromethysticin, or a derivative thereof and at least three other styrylpyrones.

In one embodiment, the composition promotes the expression of four or more chalcones.

In one embodiment, the four or more chalcones are four or more of the group comprising or consisting of pinocembrin chalcone, naringenin chalcone, flavokavain A, flavokavain B, and flavokavain C, or derivatives thereof.

In one embodiment, the four or more chalcones are flavokavain A, flavokavain B, flavokavain C, or derivatives thereof and at least one other chalcone

In one embodiment, the four or more chalcones are pinocembrin chalcone, or a derivative thereof and at least three other chalcones.

In one embodiment, the four or more chalcones are naringenin chalcone, or a derivative thereof and at least three other chalcones.

In one embodiment, the four or more chalcones are flavokavain A, or a derivative thereof and at least three other chalcones.

In one embodiment, the four or more chalcones are flavokavain B, or a derivative thereof and at least three other chalcones.

In one embodiment, the four or more chalcones are flavokavain C, or a derivative thereof and at least three other chalcones.

In one embodiment, the composition promotes the expression of five or more styrylpyrones and/or chalcones.

In one embodiment, the five or more styrylpyrones and/or chalcones are five or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2,trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, katsumadain C, pinocembrin chalcone, naringenin chalcone, flavokavain A, flavokavain B, and flavokavain C, or derivatives thereof.

In one embodiment, the composition promotes the expression of five or more styrylpyrones.

In one embodiment, the five or more styrylpyrones are five or more kavalactones.

In a further embodiment, the five or more kavalactones comprise five or more plant-derived kavalactones.

In one embodiment, the five or more styrylpyrones comprise five or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2, trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, and katsumadain C, or derivatives thereof.

In one embodiment, the five or more styrylpyrones comprise five or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the five or more styrylpyrones comprise five or more of the group comprising or consisting of 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the five or more styrylpyrones comprise five or more of the group comprising or consisting of kavain, 7,8-dihydrokavain, desmethoxyyangonin, yangonin, methysticin, and 7,8-dihydromethysticin, or derivatives thereof.

In one embodiment, the five or more styrylpyrones are yangonin, or a derivative thereof and at least four other styrylpyrones.

In one embodiment, the five or more styrylpyrones are kavain, or a derivative thereof and at least four other styrylpyrones.

In one embodiment, the five or more styrylpyrones are 7,8-dihydrokavain, or a derivative thereof and at least four other styrylpyrones.

In one embodiment, the five or more styrylpyrones are desmethoxyyangonin, or a derivative thereof and at least four other styrylpyrones.

In one embodiment, the five or more styrylpyrones are methysticin, or a derivative thereof and at least four other styrylpyrone.

In one embodiment, the five or more styrylpyrones are 7,8-dihydromethysticin, or a derivative thereof and at least four other styrylpyrones.

In one embodiment, the composition promotes the expression of five or more chalcones.

In one embodiment, the five or more chalcones are pinocembrin chalcone, naringenin chalcone, flavokavain A, flavokavain B, and flavokavain C, or derivatives thereof.

In one embodiment, the five or more chalcones are flavokavain A, flavokavain B, flavokavain C, or derivatives thereof and at least two other chalcones.

In one embodiment, the five or more chalcones are pinocembrin chalcone, or a derivative thereof and at least four other chalcones.

In one embodiment, the five or more chalcones are naringenin chalcone, or a derivative thereof and at least four other chalcones.

In one embodiment, the five or more chalcones are flavokavain A, or a derivative thereof and at least four other chalcones.

In one embodiment, the five or more chalcones are flavokavain B, or a derivative thereof and at least four other chalcones.

In one embodiment, the five or more chalcones are flavokavain C, or a derivative thereof and at least four other chalcones.

In one embodiment, the composition promotes the expression of 6, 7, 8, 9, 10, 11, 12, 13, 14, 15 or more styrylpyrones and/or chalcones.

In one embodiment, the 6, 7, 8, 9, 10, 11, 12, 13, 14, 15 or more styrylpyrones and/or chalcones are 6, 7, 8, 9, 10, 11, 12, 13, 14, 15 or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2, trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, katsumadain C, pinocembrin chalcone, naringenin chalcone, flavokavain A, flavokavain B, and flavokavain C, or derivatives thereof.

In one embodiment, the composition promotes the expression of 6, 7, 8, 9, 10, 11, 12, 13, 14, 15 or more styrylpyrones.

In one embodiment, the 6, 7, 8, 9, 10, 11, 12, 13, 14, 15 or more styrylpyrones are 6, 7, 8, 9, 10, 11, 12, 13, 14, 15 or more kavalactones.

In a further embodiment, the 6, 7, 8, 9, 10, 11, 12, 13, 14, 15 or more kavalactones comprise 6, 7, 8, 9, 10, 11, 12, 13, 14, 15 or more plant-derived kavalactones.

In one embodiment, the 6, 7, 8, 9, 10, 11, 12, 13, 14, 15 or more styrylpyrones comprise 6, 7, 8, 9, 10, 11, 12, 13, 14, 15 or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2, trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, and katsumadain C, or derivatives thereof.

In one embodiment, the 6, 7, 8, 9, 10, 11, 12, 13, 14, 15 or more styrylpyrones comprise 6, 7, 8, 9, 10, 11, 12, 13, 14, 15 or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the 6, 7, 8, 9, 10, 11, 12, 13, 14, 15 or more styrylpyrones comprise 6, 7, 8, 9, 10, 11, 12, 13, 14, 15 or more of the group comprising or consisting of 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the 6, 7, 8, 9, 10, 11, 12, 13, 14, 15 or more styrylpyrones are kavain, 7,8-dihydrokavain, desmethoxyyangonin, yangonin, methysticin, 7,8-dihydromethysticin, or derivatives thereof and at least 0, 1, 2, 3, 4, 5, 6, 7, 8, 9 other styrylpyrones.

In one embodiment, the 6, 7, 8, 9, 10, 11, 12, 13, 14, 15 or more styrylpyrones are yangonin, or a derivative thereof and at least 5, 6, 7, 8, 9, 10, 11, 12, 13, 14 other styrylpyrones.

In one embodiment, the 6, 7, 8, 9, 10, 11, 12, 13, 14, 15 or more styrylpyrones are kavain, or a derivative thereof and at least 5, 6, 7, 8, 9, 10, 11, 12, 13, 14 other styrylpyrones.

In one embodiment, the 6, 7, 8, 9, 10, 11, 12, 13, 14, 15 or more styrylpyrones are 7,8-dihydrokavain, or a derivative thereof and at least 5, 6, 7, 8, 9, 10, 11, 12, 13, 14 other styrylpyrones.

In one embodiment, the 6, 7, 8, 9, 10, 11, 12, 13, 14, 15 or more styrylpyrones are desmethoxyyangonin, or a derivative thereof and at least 5, 6, 7, 8, 9, 10, 11, 12, 13, 14 other styrylpyrones.

In one embodiment, the 6, 7, 8, 9, 10, 11, 12, 13, 14, 15 or more styrylpyrones are methysticin, or a derivative thereof and at least 5, 6, 7, 8, 9, 10, 11, 12, 13, 14 other styrylpyrones.

In one embodiment, the 6, 7, 8, 9, 10, 11, 12, 13, 14, 15 or more styrylpyrones are 7,8-dihydromethysticin, or a derivative thereof and at least 5, 6, 7, 8, 9, 10, 11, 12, 13, 14 other styrylpyrones.

### Method

As described herein, in one aspect there is provided a method of promoting the expression of one or more styrylpyrones and/or chalcones in a cell, comprising the step of:
a) introducing the composition according to any one of the composition embodiments described herein into a cell.

In one embodiment, the method further comprises:
b) extracting the one or more styrylpyrones and/or chalcones from the cell to produce an extract.

In an additional embodiment, the method further comprises:
c) detecting the one or more styrylpyrones and/or chalcones in the extract.

In an additional embodiment, the method further comprises:
d) purifying the one or more styrylpyrones and/or chalcones from the extract.

In one embodiment, the method further comprises one or more of:
b) extracting the one or more styrylpyrones and/or chalcones from the cell to produce an extract;
c) detecting the one or more styrylpyrones and/or chalcones in the extract; and
d) purifying the one or more styrylpyrones and/or chalcones from the extract.

In one embodiment, the method further comprises:
b) extracting the one or more styrylpyrones and/or chalcones from the cell to produce an extract;
c) detecting the one or more styrylpyrones and/or chalcones in the extract; and
d) purifying the one or more styrylpyrones and/or chalcones from the extract.

In a further embodiment, the one or more styrylpyrones and/or chalcones are purified using Flash and preparative MPLC, HPTLC or LC/MS.

In one embodiment, the cell is a plant cell, yeast cell and/or bacteria cell.

In a further embodiment, the plant cell is an algae cell, moss cell, or any cell or cell line derived from a plant.

In yet a further embodiment, the cell or cell line derived from a plant, is a cell or cell line derived from the genus Nicotiana.

In yet another further embodiment, the cell or cell line derived from the genus Nicotiana is selected from the group comprising or consisting of Nicotiana tabacum, Nicotiana benthamiana, or BY-2 cells.

In one embodiment, the composition promotes the expression of one or more styrylpyrones and/or chalcones.

In one embodiment, the one or more styrylpyrones and/or chalcones are one or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2,trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, katsumadain C, pinocembrin chalcone, naringenin chalcone, flavokavain A, flavokavain B, and flavokavain C, or derivatives thereof.

In one embodiment, the composition promotes the expression of one or more styrylpyrones.

In one embodiment, the one or more styrylpyrones are one or more kavalactones.

In a further embodiment, the one or more kavalactones comprise one or more plant-derived kavalactones.

In one embodiment, the one or more styrylpyrones comprise one or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2, trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, and katsumadain C, or derivatives thereof.

In one embodiment, the one or more styrylpyrones comprise one or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the one or more styrylpyrones comprise one or more of the group comprising or consisting of 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the one or more styrylpyrones comprise one or more of the group comprising or consisting of kavain, 7,8-dihydrokavain, desmethoxyyangonin, yangonin, methysticin, and 7,8-dihydromethysticin, or derivatives thereof.

In one embodiment, the one or more styrylpyrones is yangonin, or a derivative thereof.

In one embodiment, the one or more styrylpyrones is kavain, or a derivative thereof.

In one embodiment, the one or more styrylpyrones is 7,8-dihydrokavain, or a derivative thereof.

In one embodiment, the one or more styrylpyrones is desmethoxyyangonin, or a derivative thereof.

In one embodiment, the one or more styrylpyrones is methysticin, or a derivative thereof.

In one embodiment, the one or more styrylpyrones is 7,8-dihydromethysticin, or a derivative thereof.

In one embodiment, the composition promotes the expression of one or more chalcones.

In one embodiment, the one or more chalcones are one or more flavokavains.

In one embodiment, the one or more chalcones are one or more of the group comprising or consisting of pinocembrin chalcone, naringenin chalcone, flavokavain A, flavokavain B, and flavokavain C, or derivatives thereof.

In one embodiment, the one or more chalcones is pinocembrin chalcone, or a derivative thereof.

In one embodiment, the one or more chalcones is naringenin chalcone, or a derivative thereof.

In one embodiment, the one or more chalcones is flavokavain A, or a derivative thereof.

In one embodiment, the one or more chalcones is flavokavain B, or a derivative thereof.

In one embodiment, the one or more chalcones is flavokavain C, or a derivative thereof.

In one embodiment, the composition promotes the expression of two or more styrylpyrones and/or chalcones.

In one embodiment, the two or more styrylpyrones and/or chalcones are two or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2, trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, katsumadain C, pinocembrin chalcone, naringenin chalcone, flavokavain A, flavokavain B, and flavokavain C, or derivatives thereof.

In one embodiment, the composition promotes the expression of two or more styrylpyrones.

In one embodiment, the two or more styrylpyrones are two or more kavalactones.

In a further embodiment, the two or more kavalactones comprise two or more plant-derived kavalactones.

In one embodiment, the two or more styrylpyrones comprise two or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2,trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, and katsumadain C, or derivatives thereof.

In one embodiment, the two or more styrylpyrones comprise two or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the two or more styrylpyrones comprise two or more of the group comprising or consisting of 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the two or more styrylpyrones comprise two or more of the group comprising or consisting of kavain, 7,8-dihydrokavain, desmethoxyyangonin, yangonin, methysticin, and 7,8-dihydromethysticin, or derivatives thereof.

In one embodiment, the two or more styrylpyrones are yangonin, or a derivative thereof and at least one other styrylpyrone.

In one embodiment, the two or more styrylpyrones are kavain, or a derivative thereof and at least one other styrylpyrone.

In one embodiment, the two or more styrylpyrones are 7,8-dihydrokavain, or a derivative thereof and at least one other styrylpyrone.

In one embodiment, the two or more styrylpyrones are desmethoxyyangonin, or a derivative thereof and at least one other styrylpyrone.

In one embodiment, the two or more styrylpyrones are methysticin, or a derivative thereof and at least one other styrylpyrone.

In one embodiment, the two or more styrylpyrones are 7,8-dihydromethysticin, or a derivative thereof and at least one other styrylpyrone.

In one embodiment, the composition promotes the expression of two or more chalcones.

In one embodiment, the two or more chalcones are two or more flavokavains.

In one embodiment, the two or more chalcones are two or more of the group comprising or consisting of pinocembrin chalcone, naringenin chalcone, flavokavain A, flavokavain B, and flavokavain C, or derivatives thereof.

In one embodiment, the two or more chalcones are pinocembrin chalcone, or a derivative thereof and at least one other chalcone.

In one embodiment, the two or more chalcones are naringenin chalcone, or a derivative thereof and at least one other chalcone.

In one embodiment, the two or more chalcones are flavokavain A, or a derivative thereof and at least one other chalcone.

In one embodiment, the two or more chalcones are flavokavain B, or a derivative thereof and at least one other chalcone.

In one embodiment, the two or more chalcones are flavokavain C, or a derivative thereof and at least one other chalcone.

In one embodiment, the composition promotes the expression of three or more styrylpyrones and/or chalcones.

In one embodiment, the three or more styrylpyrones and/or chalcones are three or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2, trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, katsumadain C, pinocembrin chalcone, naringenin chalcone, flavokavain A, flavokavain B, and flavokavain C, or derivatives thereof.

In one embodiment, the composition promotes the expression of three or more styrylpyrones.

In one embodiment, the three or more styrylpyrones are three or more kavalactones.

In a further embodiment, the three or more kavalactones comprise three or more plant-derived kavalactones.

In one embodiment, the three or more styrylpyrones comprise three or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2, trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, and katsumadain C, or derivatives thereof.

In one embodiment, the three or more styrylpyrones comprise three or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the three or more styrylpyrones comprise three or more of the group comprising or consisting of 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the three or more styrylpyrones comprise three or more of the group comprising or consisting of kavain, 7,8-dihydrokavain, desmethoxyyangonin, yangonin, methysticin, and 7,8-dihydromethysticin, or derivatives thereof.

In one embodiment, the three or more styrylpyrones are yangonin, or a derivative thereof and at least two other styrylpyrones.

In one embodiment, the three or more styrylpyrones are kavain, or a derivative thereof and at least two other styrylpyrones.

In one embodiment, the three or more styrylpyrones are 7,8-dihydrokavain, or a derivative thereof and at least two other styrylpyrones.

In one embodiment, the three or more styrylpyrones are desmethoxyyangonin, or a derivative thereof and at least two other styrylpyrones.

In one embodiment, the three or more styrylpyrones are methysticin, or a derivative thereof and at least two other styrylpyrones.

In one embodiment, the three or more styrylpyrones are 7,8-dihydromethysticin, or a derivative thereof and at least two other styrylpyrones.

In one embodiment, the composition promotes the expression of three or more chalcones.

In one embodiment, the three or more chalcones are three or more of the group comprising or consisting of pinocembrin chalcone, naringenin chalcone, flavokavain A, flavokavain B, and flavokavain C, or derivatives thereof.

In one embodiment, the three or more chalcones are flavokavain A, flavokavain B, and flavokavain C, or derivatives thereof.

In one embodiment, the three or more chalcones are pinocembrin chalcone, or a derivative thereof and at least two other chalcones.

In one embodiment, the three or more chalcones are naringenin chalcone, or a derivative thereof and at least two other chalcones.

In one embodiment, the three or more chalcones are flavokavain A, or a derivative thereof and at least two other chalcones.

In one embodiment, the three or more chalcones are flavokavain B, or a derivative thereof and at least two other chalcones.

In one embodiment, the three or more chalcones are flavokavain C, or a derivative thereof and at least two other chalcones.

In one embodiment, the composition promotes the expression of four or more styrylpyrones and/or chalcones.

In one embodiment, the four or more styrylpyrones and/or chalcones are four or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2, trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, katsumadain C, pinocembrin chalcone, naringenin chalcone, flavokavain A, flavokavain B, and flavokavain C, or derivatives thereof.

In one embodiment, the composition promotes the expression of four or more styrylpyrones.

In one embodiment, the four or more styrylpyrones are four or more kavalactones.

In a further embodiment, the four or more kavalactones comprise four or more plant-derived kavalactones.

In one embodiment, the four or more styrylpyrones comprise four or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2,trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, and katsumadain C, or derivatives thereof.

In one embodiment, the four or more styrylpyrones comprise four or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the four or more styrylpyrones comprise four or more of the group comprising or consisting of 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the four or more styrylpyrones comprise four or more of the group comprising or consisting of kavain, 7,8-dihydrokavain, desmethoxyyangonin, yangonin, methysticin, and 7,8-dihydromethysticin, or derivatives thereof.

In one embodiment, the four or more styrylpyrones are yangonin, or a derivative thereof and at least three other styrylpyrones.

In one embodiment, the four or more styrylpyrones are kavain, or a derivative thereof and at least three other styrylpyrones.

In one embodiment, the four or more styrylpyrones are 7,8-dihydrokavain, or a derivative thereof and at least three other styrylpyrones.

In one embodiment, the four or more styrylpyrones are desmethoxyyangonin, or a derivative thereof and at least three other styrylpyrones.

In one embodiment, the four or more styrylpyrones are methysticin, or a derivative thereof and at least three other styrylpyrones.

In one embodiment, the four or more styrylpyrones are 7,8-dihydromethysticin, or a derivative thereof and at least three other styrylpyrones.

In one embodiment, the composition promotes the expression of four or more chalcones.

In one embodiment, the four or more chalcones are four or more of the group comprising or consisting of pinocembrin chalcone, naringenin chalcone, flavokavain A, flavokavain B, and flavokavain C, or derivatives thereof.

In one embodiment, the four or more chalcones are flavokavain A, flavokavain B, flavokavain C, or derivatives thereof and at least one other chalcone.

In one embodiment, the four or more chalcones are pinocembrin chalcone, or a derivative thereof and at least three other chalcones.

In one embodiment, the four or more chalcones are naringenin chalcone, or a derivative thereof and at least three other chalcones.

In one embodiment, the four or more chalcones are flavokavain A, or a derivative thereof and at least three other chalcones.

In one embodiment, the four or more chalcones are flavokavain B, or a derivative thereof and at least three other chalcones.

In one embodiment, the four or more chalcones are flavokavain C, or a derivative thereof and at least three other chalcones.

In one embodiment, the composition promotes the expression of five or more styrylpyrones and/or chalcones.

In one embodiment, the five or more styrylpyrones and/or chalcones are five or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2,trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, katsumadain C, pinocembrin chalcone, naringenin chalcone, flavokavain A, flavokavain B, and flavokavain C, or derivatives thereof.

In one embodiment, the composition promotes the expression of five or more styrylpyrones.

In one embodiment, the five or more styrylpyrones are five or more kavalactones.

In a further embodiment, the five or more kavalactones comprise five or more plant-derived kavalactones.

In one embodiment, the five or more styrylpyrones comprise five or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2, trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, and katsumadain C, or derivatives thereof.

In one embodiment, the five or more styrylpyrones comprise five or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the five or more styrylpyrones comprise five or more of the group comprising or consisting of 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the five or more styrylpyrones comprise five or more of the group comprising or consisting of kavain, 7,8-dihydrokavain, desmethoxyyangonin, yangonin, methysticin, and 7,8-dihydromethysticin, or derivatives thereof.

In one embodiment, the five or more styrylpyrones are yangonin, or a derivative thereof and at least four other styrylpyrones.

In one embodiment, the five or more styrylpyrones are kavain, or a derivative thereof and at least four other styrylpyrones.

In one embodiment, the five or more styrylpyrones are 7,8-dihydrokavain, or a derivative thereof and at least four other styrylpyrones.

In one embodiment, the five or more styrylpyrones are desmethoxyyangonin, or a derivative thereof and at least four other styrylpyrones.

In one embodiment, the five or more styrylpyrones are methysticin, or a derivative thereof and at least four other styrylpyrone.

In one embodiment, the five or more styrylpyrones are 7,8-dihydromethysticin, or a derivative thereof and at least four other styrylpyrones.

In one embodiment, the composition promotes the expression of five or more chalcones.

In one embodiment, the five or more chalcones are pinocembrin chalcone, naringenin chalcone, flavokavain A, flavokavain B, and flavokavain C, or derivatives thereof.

In one embodiment, the five or more chalcones are flavokavain A, flavokavain B, flavokavain C, or derivatives thereof and at least two other chalcones.

In one embodiment, the five or more chalcones are pinocembrin chalcone, or a derivative thereof and at least four other chalcones.

In one embodiment, the five or more chalcones are naringenin chalcone, or a derivative thereof and at least four other chalcones.

In one embodiment, the five or more chalcones are flavokavain A, or a derivative thereof and at least four other chalcones.

In one embodiment, the five or more chalcones are flavokavain B, or a derivative thereof and at least four other chalcones.

In one embodiment, the five or more chalcones are flavokavain C, or a derivative thereof and at least four other chalcones.

In one embodiment, the composition promotes the expression of 6, 7, 8, 9, 10, 11, 12, 13, 14, 15 or more styrylpyrones and/or chalcones.

In one embodiment, the 6, 7, 8, 9, 10, 11, 12, 13, 14, 15 or more styrylpyrones and/or chalcones are 6, 7, 8, 9, 10, 11, 12, 13, 14, 15 or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2,trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, katsumadain C, pinocembrin chalcone, naringenin chalcone, flavokavain A, flavokavain B, and flavokavain C, or derivatives thereof.

In one embodiment, the composition promotes the expression 6, 7, 8, 9, 10, 11, 12, 13, 14, 15 or more styrylpyrones.

In one embodiment, the 6, 7, 8, 9, 10, 11, 12, 13, 14, 15 or more styrylpyrones are 6, 7, 8, 9, 10, 11, 12, 13, 14, 15 or more kavalactones.

In a further embodiment, the 6, 7, 8, 9, 10, 11, 12, 13, 14, 15 or more kavalactones comprise 6, 7, 8, 9, 10, 11, 12, 13, 14, 15 or more plant-derived kavalactones.

In one embodiment, the 6, 7, 8, 9, 10, 11, 12, 13, 14, 15 or more styrylpyrones comprise 6, 7, 8, 9, 10, 11, 12, 13, 14, 15 or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2, trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, and katsumadain C, or derivatives thereof.

In one embodiment, the 6, 7, 8, 9, 10, 11, 12, 13, 14, 15 or more styrylpyrones comprise 6, 7, 8, 9, 10, 11, 12, 13, 14, 15 or more of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the 6, 7, 8, 9, 10, 11, 12, 13, 14, 15 or more styrylpyrones comprise 6, 7, 8, 9, 10, 11, 12, 13, 14, 15 or more of the group comprising or consisting of 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or derivatives thereof.

In one embodiment, the 6, 7, 8, 9, 10, 11, 12, 13, 14, 15 or more styrylpyrones are kavain, 7,8-dihydrokavain, desmethoxyyangonin, yangonin, methysticin, 7,8-dihydromethysticin, or derivatives thereof and at least 0, 1, 2, 3, 4, 5, 6, 7, 8, 9 other styrylpyrones.

In one embodiment, the 6, 7, 8, 9, 10, 11, 12, 13, 14, 15 or more styrylpyrones are yangonin, or a derivative thereof and at least 5, 6, 7, 8, 9, 10, 11, 12, 13, 14 other styrylpyrones.

In one embodiment, the 6, 7, 8, 9, 10, 11, 12, 13, 14, 15 or more styrylpyrones are kavain, or a derivative thereof and at least 5, 6, 7, 8, 9, 10, 11, 12, 13, 14 other styrylpyrones.

In one embodiment, the 6, 7, 8, 9, 10, 11, 12, 13, 14, 15 or more styrylpyrones are 7,8-dihydrokavain, or a derivative thereof and at least 5, 6, 7, 8, 9, 10, 11, 12, 13, 14 other styrylpyrones.

In one embodiment, the 6, 7, 8, 9, 10, 11, 12, 13, 14, 15 or more styrylpyrones are desmethoxyyangonin, or a derivative thereof and at least 5, 6, 7, 8, 9, 10, 11, 12, 13, 14 other styrylpyrones.

In one embodiment, the 6, 7, 8, 9, 10, 11, 12, 13, 14, 15 or more styrylpyrones are methysticin, or a derivative thereof and at least 5, 6, 7, 8, 9, 10, 11, 12, 13, 14 other styrylpyrones.

In one embodiment, the 6, 7, 8, 9, 10, 11, 12, 13, 14, 15 or more styrylpyrones are 7,8-dihydromethysticin, or a derivative thereof and at least 5, 6, 7, 8, 9, 10, 11, 12, 13, 14 other styrylpyrones.

### Products

As described herein, in one aspect there is provided a styrylpyrone or chalcone obtainable from the method according to any one of the method embodiments described herein.

In one embodiment, the styrylpyrone or chalcone is any one of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2,trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, katsumadain C, pinocembrin chalcone, naringenin chalcone, flavokavain A, flavokavain B, and flavokavain C, or a derivative thereof.

In one embodiment, there is provided a styrylpyrone obtainable from the method according to any one of the method embodiments described herein.

In one embodiment, the styrylpyrone is a kavalactone.

In a further embodiment, the kavalactone is a plant-derived kavalactone.

In one embodiment, the styrylpyrone is any one of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, Yangonin, 11-methoxyangonin, 10-methoxyangonin, 11-hydroxyangonin, 11-methoxy-12-hydroxydehydrokavain, 11-methoxy-5,6-dihydroyangonin, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, 4-methoxy-6 (11,-12-methylenedioxy-10,14-dimethoxydihydrostyryl)-2-pyrone, 4-methoxy-6-(11,12-methylenedioxy-14-methoxystyryl)-2-pyrone, 4-methoxy-6-(11,12-methylenedioxystyryl)-2-pyrone, 4-methoxy-6-(11,12-dimethylstyryl)-2-pyrone, 7,8-epoxyyangonin, (+)-Goniothalamin, goniodiol, (+)-Altholactone, (+)-garvensintriol, (+)-etharvendiol, (−)-etharvensin, goniotriol, Dumortins A, Dumortins B, Dumortins C, Diyangonins A, Diyangonins B, rel-trans-3-bis[6-(4-methoxy-2-pyronyl)]-cis-2, trans-4-diphenyl cyclobutane, Diyangonins C, 6,6′-(3,4-diphenylcyclobutane-1,2-diyl)bis(4-methoxy-2H-pyran-2-one), Yangonindimers A, Yangonindimers B, Yangonindimers C, aniba-dimer A, (r-8,c-7′,t-7-8,8′-11,12-dimethoxyphenyl-7,7′-di-[6-(4-methoxy-2-pyronyl)]-cyclobutane, (rel-(6R,7S,8S,5'S)-4′-methoxy-8-(11,12-dimethoxyphenyl)-7-[6-(4-methoxy-2-pyronyl)]-6-(E)-styryl-1′-oxabicyclo[4,2,0]octa-4′-en-2′-one, katsumadain A, katsumadain B, and katsumadain C, or a derivative thereof.

In one embodiment, the styrylpyrone is any one of the group comprising or consisting of 4-hydroxy-6-styryl-2-pyrone C7=C8, bisnoryangonin C7=C8, 7,8-dihydrobisnoryangonin C7-C8, hispidin C7=C8, 11-methoxy-bisnoryangonin C7=C8, 10-hydroxy-bisnoryangonin, 4-hydroxy-6-phenethyl-2-pyrone C7-C8, 7,8-dihydrohispidin C7-C8, 11-methoxy-7,8-dihydrobisnoryangoin C7-C8, 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or a derivative thereof.

In one embodiment, the styrylpyrone is any one of the group comprising or consisting of 11-hydroxy-12-methoxydihydrokavain, 7,8-dihydrokavain, 7,8-dihydromethysticin, 5,6,7,8-tetrahydroyangonin, 11,12-dimethoxy-5,6-dihydrokavain, 7,8-dihydro-5-hydroxykavain, kavain, methysticin, 5,6-dihydroyangonin, 5-hydroxykavain, 5,6-dehydromethysticin, 7,8-dihydroyangonin, desmethoxyyangonin, yangonin, 11-methoxyangonin, 10-methoxyangonin, 11-hydroxyangonin, 11-methoxy-12-hydroxydehydrokavain, 11-methoxy-5,6-dihydroyangonin, and 7,8-dihydro-5,6-dehydrokavain, or a derivative thereof.

In one embodiment, the styrylpyrone is any one of the group comprising or consisting of kavain, 7,8-dihydrokavain, desmethoxyyangonin, yangonin, methysticin, and 7,8-dihydromethysticin, or a derivative thereof.

In one embodiment, the styrylpyrone is yangonin, or a derivative thereof.

In one embodiment, the styrylpyrone is kavain, or a derivative thereof.

In one embodiment, the styrylpyrone is 7,8-dihydrokavain, or a derivative thereof.

In one embodiment, the styrylpyrone is desmethoxyyangonin, or a derivative thereof.

In one embodiment, the styrylpyrone is methysticin, or a derivative thereof.

In one embodiment, the styrylpyrone is 7,8-dihydromethysticin, or a derivative thereof.

In one embodiment, there is provided a chalcone obtainable from the method according to any one of the method embodiments described herein.

In one embodiment, the chalcone is a flavokavain.

In one embodiment, the chalcone is any one of the group comprising or consisting of pinocembrin chalcone, naringenin chalcone, flavokavain A, flavokavain B, and flavokavain C, or a derivative thereof.

In one embodiment, the chalcone is pinocembrin chalcone, or a derivative thereof.

In one embodiment, the chalcone is naringenin chalcone, or a derivative thereof.

In one embodiment, the chalcone is flavokavain A, or a derivative thereof.

In one embodiment, the chalcone is flavokavain B, or a derivative thereof.

In one embodiment, the chalcone is flavokavain C, or a derivative thereof.

### Vector Combinations

As described herein, different combinations of enzymes present in the styrylpyrone or flavokavain pathway and transcription factors present in the flavonoid pathway result in increased expression of different styrylpyrones and/or chalcones.

There now follows non-limiting examples of different compositions designed to promote the expression of one or more styrylpyrones and/or chalcones.

In one embodiment, there is provided a composition for promoting the expression of one or more styrylpyrones and/or chalcones in a cell, wherein the composition comprises:
a) one or more first vector components encoding SPS1; and
b) one or more second vector components encoding MYB12, DEL, and ROS1.

In a further embodiment, the composition comprising one or more first and second vector components encoding SPS1, MYB12, DEL, and ROS1 promotes the expression of 4-hydroxy-6-styryl-2-pyrone C7=C8, 11-methoxy-bisnoryangonin C7=C8, 10-hydroxy-bisnoryangonin, 4-hydroxy-6-phenethyl-2-pyrone C7-C8, and 11-methoxy-7,8-dihydrobisnoryangoin C7-C8.

In one embodiment, there is provided a composition for promoting the expression of one or more styrylpyrones and/or chalcones in a cell, wherein the composition comprises:
a) one or more first vector components encoding SPS2; and
b) one or more second vector components encoding MYB12, DEL, and ROS1.

In a further embodiment, the composition comprising one or more first and second vector components encoding SPS2, MYB12, DEL, and ROS1 promotes the expression of 4-hydroxy-6-styryl-2-pyrone C7=C8, bisnoryangonin C7=C8, 7,8-dihydrobisnoryangonin C7-C8, hispidin C7=C8, 4-hydroxy-6-phenethyl-2-pyrone C7-C8, and 7,8-dihydrohispidin C7-C8.

In one embodiment, there is provided a composition for promoting the expression of one or more styrylpyrones and/or chalcones in a cell, wherein the composition comprises:
a) one more first vector components encoding KLR1, KOMT1, SPS1; and
b) one or more second vector components encoding MYB12, DEL, and ROS1.

In a further embodiment, the composition comprising one or more first and second vector components encoding KLR1, KOMT1, SPS1, MYB12, DEL, and ROS1 promotes the expression of 11-hydroxy-12-methoxydihydrokavain, 7,8-dihydrokavain, 11,12-dimethoxy-5,6-dihydrokavain, kavain, and 11-methoxy-5,6-dihydroyangonin.

In one embodiment, there is provided a composition for promoting the expression of one or more styrylpyrones and/or chalcones in a cell, wherein the composition comprises:
a) one or more first vector components encoding KLR1, KOMT1, SPS2; and
b) one or more second vector components encoding MYB12, DEL, and ROS1.

In a further embodiment, the composition comprising one or more first and second vector components encoding KLR1, KOMT1, SPS2, MYB12, DEL, and ROS1 promotes the expression of 11-hydroxy-12-methoxydihydrokavain, 7,8-dihydrokavain, 5,6,7,8-tetrahydroyangonin, 11,12-dimethoxy-5,6-dihydrokavain, kavain, 5,6-dihydroyangonin, and 11-methoxy-5,6-dihydroyangonin.

In one embodiment, there is provided a composition for promoting the expression of one or more styrylpyrones and/or chalcones in a cell, wherein the composition comprises:
a) one or more first vector components encoding KLR1, KOMT1, CYP719A26, SPS2; and
b) one or more second vector components encoding MYB12, DEL, and ROS1.

In a further embodiment, the composition comprising one or more first and second vector components encoding KLR1, KOMT1, CYP719A26, SPS2, MYB12, DEL, and ROS1 promotes the expression of 7,8-dihydromethysticin, and methysticin.

In one embodiment, there is provided a composition for promoting the expression of one or more styrylpyrones and/or chalcones in a cell, wherein the composition comprises:
a) one or more first vector components encoding KLR1, P450, KOMT1, SPS1; and
b) one or more second vector components encoding MYB12, DEL, and ROS1.

In a further embodiment, the composition comprising one or more first and second vector components encoding KLR1, P450, KOMT1, SPS1, MYB12, DEL, and ROS1 promotes the expression of 7,8-dihydro-5-hydroxykavain, and 5-hydroxykavain.

In one embodiment, there is provided a composition for promoting the expression of one or more styrylpyrones and/or chalcones in a cell, wherein the composition comprises:
a) one or more first vector components encoding KLR1, P450, KOMT1, SPS2; and
b) one or more second vector components encoding MYB12, DEL, and ROS1.

In a further embodiment, the composition comprising one or more first and second vector components encoding KLR1, P450, KOMT1, SPS2, MYB12, DEL, and ROS1 promotes the expression of 7,8-dihydro-5-hydroxykavain, and 5-hydroxykavain.

In one embodiment, there is provided a composition for promoting the expression of one or more styrylpyrones and/or chalcones in a cell, wherein the composition comprises:
a) one or more first vector components encoding KOMT1, CYP719A26, SPS1; and
b) one or more second vector components encoding MYB12, DEL, and ROS1.

In a further embodiment, the composition comprising one or more first and second vector components encoding KOMT1, CYP719A26, SPS1, MYB12, DEL, and ROS1 promotes the expression of 5,6-dehydromethysticin.

In one embodiment, there is provided a composition for promoting the expression of one or more styrylpyrones and/or chalcones in a cell, wherein the composition comprises:
a) one or more first vector components encoding KOMT1, CYP719A26, SPS2; and
b) one or more second vector components encoding MYB12, DEL, and ROS1.

In a further embodiment, the composition comprising one or more first and second vector components encoding KOMT1, CYP719A26, SPS2, MYB12, DEL, and ROS1 promotes the expression of 5,6-dehydromethysticin.

In one embodiment, there is provided a composition for promoting the expression of one or more styrylpyrones and/or chalcones in a cell, wherein the composition comprises:
a) one or more first vector components encoding KOMT1, SPS1; and
b) one or more second vector components encoding MYB12, DEL, and ROS1.

In a further embodiment, the composition comprising one or more first and second vector components encoding KOMT1, SPS1, MYB12, DEL, and ROS1 promotes the expression of 7,8-dihydroyangonin, desmethoxyyangonin, yangonin, 11-methoxy-12-hydroxydehydrokavain, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, and 4-methoxy-6 (11,-12-methylenedioxy-10,14-dimethoxydihydrostyryl)-2-pyrone.

In one embodiment, there is provided a composition for promoting the expression of one or more styrylpyrones and/or chalcones in a cell, wherein the composition comprises:
a) one or more first vector components encoding KOMT1, SPS2; and
b) one or more second vector components encoding MYB12, DEL, and ROS1.

In a further embodiment, the composition comprising one or more first and second vector components encoding KOMT1, SPS2, MYB12, DEL, and ROS1 promotes the expression of 7,8-dihydroyangonin, desmethoxyyangonin, yangonin, 11-methoxyangonin, 11-hydroxyangonin, 11-methoxy-12-hydroxydehydrokavain, 7,8-dihydro-5,6-dehydrokavain, 4-methoxy-6-(11,12-methylenedioxydihydrostyryl)-2-pyrone, 4-methoxy-6-(11,12-methylenedioxy-14-methoxy-dihydrostyryl)-2-pyrone, and 4-methoxy-6 (11,-12-methylenedioxy-10,14-dimethoxydihydrostyryl)-2-pyrone.

In one embodiment, there is provided a composition for promoting the expression of one or more styrylpyrones and/or chalcones in a cell, wherein the composition comprises:
a) one or more first vector components encoding KOMT1, KOMT2, SPS1; and
b) one or more second vector components encoding MYB12, DEL, and ROS1.

In a further embodiment, the composition comprising one or more first and second vector components encoding KOMT1, KOMT2, SPS1, MYB12, DEL, and ROS1 promotes the expression of 10-methoxyangonin, 4-methoxy-6-(11,12-methylenedioxy-14-methoxystyryl)-2-pyrone, 4-methoxy-6-(11,12-methylenedioxystyryl)-2-pyrone, and 4-methoxy-6-(11,12-dimethylstyryl)-2-pyrone.

In one embodiment, there is provided a composition for promoting the expression of one or more styrylpyrones and/or chalcones in a cell, wherein the composition comprises:
a) one or more first vector components encoding CHS; and
b) one or more second vector components encoding MYB12, DEL, and ROS1.

In a further embodiment, the composition comprising one or more first and second vector components encoding CHS, MYB12, DEL, and ROS1 promotes the expression of pinocembrin chalcone and naringenin chalcone.

In one embodiment, there is provided a composition for promoting the expression of one or more styrylpyrones and/or chalcones in a cell, wherein the composition comprises:
a) one or more first vector components encoding KOMT1; and
b) one or more second vector components encoding MYB12, DEL, and ROS1.

In a further embodiment, the composition comprising one or more first and second vector components encoding KOMT1, MYB12, DEL, and ROS1 promotes the expression of Flavokavain A, Flavokavain B, and Flavokavain C.

The expressed styrylpyrones and chalcones may or may not be endogenously present in the cell, cell line, yeast, bacteria, algae, moss, or plant in which the composition of the invention is used. It will be understood by the skilled person that where a particular styrylpyrone or chalcone is not endogenously produced, there is considered to be an increase in this styrylpyrone or chalcone if it can be detected after the cell, cell line, yeast, bacteria, algae, moss or plant has been transfected with the composition of the invention.

In situations where particular styrylpyrones and/or chalcones are endogenously expressed in the cell, cell line, yeast, bacteria, algae, moss, or plant before the composition of the invention is used, a 1-fold, 2-fold, 3-fold, 4-fold, 5-fold, 10-fold, 20-fold, 40-fold, 60-fold, 80-fold, 100-fold, 150-fold, 200-fold, 300-fold, 400-fold, 600-fold, 800-fold, 1000-fold, or more increase in that particular styrylpyrone or chalcone may be observed.

The total amount of styrylpyrones and/or chalcones present in a cell, cell line, yeast, bacteria, algae, moss, or plant in which the composition of the invention is used may be increased by 1-fold, 2-fold, 3-fold, 4-fold, 5-fold, 10-fold, 20-fold, 40-fold, 60-fold, 80-fold, 100-fold, 150-fold, 200-fold, 250-fold, or more.

The various aspects and/or embodiments described herein are presented only to assist in understanding and teaching the claimed features. These aspects and/or embodiments are provided as a representative sample of aspects and/or embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc, other than those specifically described herein. In addition, this disclosure may include other inventions not presently claimed, but which may be claimed in future.

The invention will now be described with reference to the following non-limiting examples.

### EXAMPLES

### Example 1: Promoting Expression of Yangonin in Nicotiana benthamiana

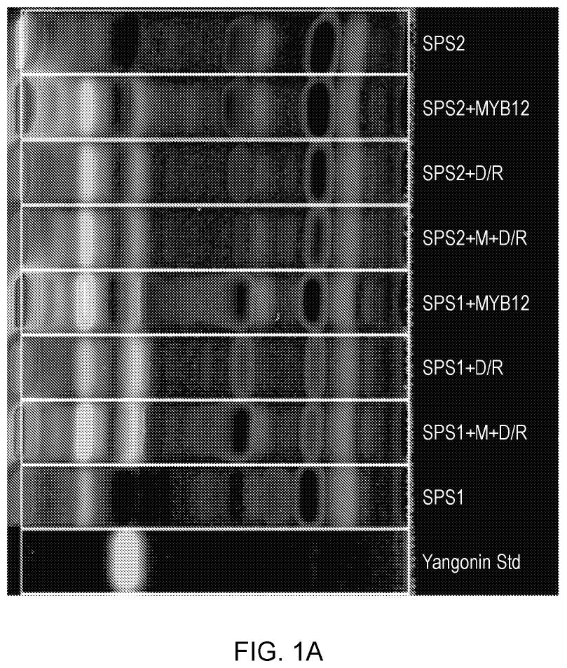

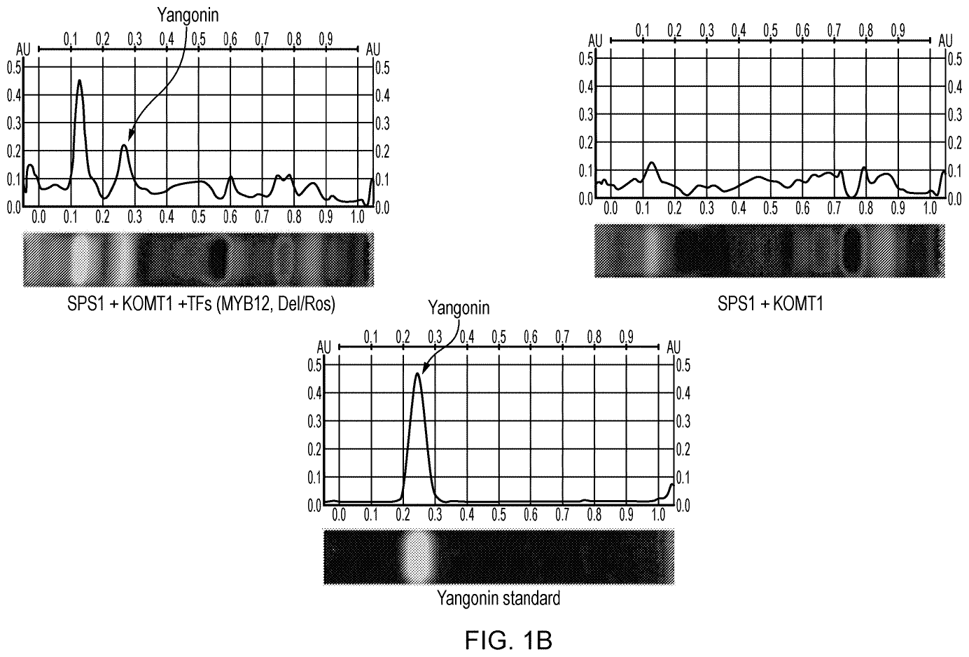

Plasmids comprising combinations of SPS1, SPS2, KOMT1, AmDEL, AmROS and AtMYB12 were generated and used to transfect Nicotiana benthamiana. The combinations were as set out in table 2 below, which corresponds to the HPTLC results in FIG. 1.

TABLE 2
Row 2: Enzyme; Row 3: Transcription Factor

| Set 1 | Set 2 | Set 3 | Set 4 | Set 5 | Set 6 | Set 7 | Set 8 |
|---|---|---|---|---|---|---|---|
| KOMT1 | KOMT1 | KOMT1 | KOMT1 | KOMT1 | KOMT1 | KOMT1 | KOMT1 |
| SPS2 | SPS2 | SPS2 | SPS2 | SPS1 | SPS1 | SPS1 | SPS1 |
| | AtMYB12 | AmDEL | AtMYB12 | AtMYB12 | AmDEL | AtMYB12 | |
| | | AmROS1 | AmDEL | | AmROS1 | AmDEL | |
| | | | AmROS1 | | | AmROS1 | |

An Agrobacterium culture (Agrobacterium tumefaciens) was used to deliver the genetic material into the Nicotiana benthamiana cells. The plasmids containing the genes of interest were introduced into separate Agrobacterium cultures and cultured to a specific density.

Once the specific density was reached (OD600 of ~1-2), it was resuspended at a density of OD600 0.2 and prepared as an infiltration solution. Infiltration solutions were prepared for each of the plasmid types (see table 4).

The infiltration solutions were then individually infiltrated into the leaves of the Nicotiana benthamiana plants, using a syringe or vacuum infiltration. Separate plants were used for each set.

Following infiltration, the plants were incubated under controlled conditions (22° C., ~250 μmol/m2/s (light), 70% humidity and 16 hr day/night cycle.) in a growth chamber for 7 days. Following this, the infiltrated leaves were frozen instantly.

The dried leaves were then ground and extraction was preformed using methanol. Extracts were then filtered and placed in vials to be analysed by HPTLC. Stationary Phase, Merck, HPTLC Silica gel 60 F₂₅₄, Mobile phase, Chloroform:Methanol 9:1 (v/v), Chloroform:Methanol:Water 80:20:2 (v/v/v), Chloroform:Methanol:Water 61:32:5 (v/v/v), Toluene:Ethyl acetate:Acetic acid:Chloroform 5:5:1:2 (v/v/v/v). After separation, the plates were derivatized with 10% H2SO4 in methanol and then visualized by normal light or UV lamp.

It was determined that set 6 and set 7, representing combinations of KOMT1, SPS1, AmDEL, & AmROS1 and KOMT1, SPS1, AtMYB12, AmDEL, & AmROS1 respectively, generated the highest yields of yangonin, whilst sets 2-5 also generated yangonin but to a lesser extent. Sets 1 and 8, which excluded the addition of transcription factors present in the flavonoid pathway and comprised KOMT1 with either SPS1 or SPS2, failed to generate yangonin, demonstrating that the addition of transcription factors present in the flavonoid pathway are important.

We found that increasing the number of (flavonoid) transcription factors encoded by the vector components, used to transfect the cell, the greater the yield of yangonin. It would be expected that transcription factors present in the flavonoid pathway would promote flavonoid expression, driving the cell to favour the flavonoid pathway over the styrylpyrone pathway, thus leading to increased expression of one or more flavonoid compounds. Therefore, it was surprising that the addition of transcription factors, and in particular the addition of multiple transcription factors, present in the flavonoid pathway were able to promote the expression of yangonin.

1. A composition for promoting the expression of one or more styrylpyrones and/or chalcones in a cell, wherein the composition comprises:
a) one or more first vector components encoding one or more enzymes present in the styrylpyrone or flavokavain pathway; and
b) one or more second vector components encoding one or more transcription factors present in the flavonoid pathway.

2. The composition according to claim 1, wherein the one or more first vector components are provided in a first vector and the one or more second vector components are provided in a second vector.

3. The composition according to claim 1, wherein the one or more first vector components and the one or more second vector components are provided in a single vector.

4. The composition according to any one of claims 1-3, wherein the one or more styrylpyrones and/or chalcones are one or more styrylpyrones.

5. The composition according to claim 4, wherein the one or more styrylpyrones are one or more kavalactones.

6. The composition according to any one of claims 1-5, wherein the one or more kavalactones comprise one or more plant-derived kavalactones.

7. The composition according to any one or claims 1-3, wherein the one or more styrylpyrones and/or chalcones are one or more chalcones.

8. The composition according to claim 7, wherein the one or more chalcones are one or more flavokavains.

9. The composition according to any one of claims 1-8, wherein the one or more enzymes present in the kavalactone or flavokavain pathways comprise styrylpyrone biosynthesis (SPS) and KOMT1.

10. The composition according to claim 9, wherein SPS is SPS1 or SPS2.

11. The composition according to claim 9 or claim 10, wherein SPS is SPS1.

12. The composition according any one of claims 1-11, wherein the one or more transcription factors present in the flavonoid pathway are independently derived from a plant species.

13. The composition according to claim 12, wherein the plant species is selected from the group comprising At (Arabidopsis thaliana), Am (Antirrhinum majus), Nt (Nicotiana tabacum), Mt (Medicago truncatula), Vv (Vitis vinifera), or Cs (Camellia sinensis).

14. A plant, plant part or plant cell transformed with vector components according to the composition of any one of claims 1-13.

15. The plant, plant part or plant cell according to claim 14, wherein the plant, plant part or plant cell is algae, moss or any plant, plant part or plant cell derived from the genus Nicotiana.

16. The plant, plant part or plant cell according to claim 15, wherein the plant, plant part or plant cell derived from the genus Nicotiana is selected from the group comprising Nicotiana tabacum, Nicotiana benthamiana, or BY-2 cells.

17. Use of the composition according to any one of claims 1-13 for promoting the expression of one or more styrylpyrones and/or chalcones in algae, moss, or any plant, plant part, or plant cells.

18. Use of the composition according claim 17, wherein the plant cells are a cell line derived from plants.

19. Use of the composition according to claim 17 or claim 18, wherein the plant, plant part, or plant cells are derived from the genus Nicotiana.

20. Use of the composition according to claim 19, the plant, plant part or plant cells derived from the genus Nicotiana are selected from the group comprising Nicotiana tabacum, Nicotiana benthamiana, or BY-2 cells.

21. Use of the composition according to any one of claims 17-20, wherein the one or more styrylpyrones and/or chalcones comprise one or more styrylpyrones.

22. Use of the composition according to claim 21, wherein the one or more styrylpyrones are one or more kavalactones.

23. Use of the composition according to claim 22, wherein the one or more kavalactones comprise one or more plant-derived kavalactones.

24. Use of the composition according to any one of claims 17-20, wherein the one or more styrylpyrones and/or chalcones comprise one or more chalcones.

25. Use of the composition according to claim 24, wherein the one or more chalcones comprise one or more flavokavains.

26. A method of promoting the expression of one or more styrylpyrones and/or chalcones in a cell, comprising the step of:
a) introducing the composition according to any one of claims 1-13 into a cell.

27. The method according to claim 26, wherein the method further comprises:
b) extracting the one or more styrylpyrones and/or chalcones from the cell to produce an extract;
c) detecting the one or more styrylpyrones and/or chalcones in the extract; and
d) purifying the one or more styrylpyrones and/or chalcones from the extract.

28. The method according to claim 27, wherein the one or more styrylpyrones and/or chalcones are purified using Flash and preparative MPLC, HPTLC or LC/MS.

29. The method according to any one of claims 26-28, wherein the cell is a plant cell.

30. The method according to claim 29, wherein the plant cell is an algae cell, moss cell, or any cell or cell line derived from a plant.

31. The method according to claim 30, wherein the cell or cell line derived from a plant is a cell or cell line derived from a plant of the genus Nicotiana.

32. The method according to claim 31, wherein the cell or cell line derived from the genus Nicotiana is selected from the group comprising Nicotiana tabacum, Nicotiana benthamiana, or BY-2 cells.

33. The method according to any one of claims 26-32, wherein the one or more styrylpyrones and/or chalcones comprise one or more styrylpyrones.

34. The method according to claim 33, wherein the one or more styrylpyrones comprise one or more kavalactones.

35. The method according to claim 34, wherein the one or more kavalactones comprise one or more plant-derived kavalactones.

36. The method according to any one of claims 26-32, wherein the one or more styrylpyrones and/or chalcones comprise one or more chalcones.

37. The method according to claim 36, wherein the one or more chalcones comprise one or more flavokavains.

38. A styrylpyrone obtainable from the method according to any one of claims 26-33.

39. The styrylpyrone according to claim 38, wherein the styrylpyrone is a kavalactone.

40. The kavalactone according to claim 39, wherein the kavalactone is a plant-derived kavalactone.

41. A chalcone obtainable from the method according to any one of claims 26-32 and 36.

42. The chalcone according to claim 41, wherein the chalcone is a flavokavain.


SEQUENCE LISTING (6 sequences)

SEQ ID NO: 1 - DNA, 17068 bp, synthetic construct
  source 1..17068: mol_type = other DNA
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgacatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacctcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcggggaggcgcgcctgatcagcttgcatgccggtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatggcaagaccggcaacaggattcaatcttaagaaactttattgccaaatgtttgaacgatctgcttgactctagctagagtccgaaccccagagtcccgctcagaagaactcgtcaagaaggcgatagaaggctatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcgtcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcggtgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctcatgtataggcgcgcctatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggcgtgtggaattgtgagcggataacaatttcacacaggaaacagctatgaccatgattacggtaccgggcccgaattgacgcgtattgggattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggctatcgttcaagatgcctctaccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgcccgggatggctactggtatccaaaaccaaaagatagtgcctgagaatttgaggaagcaacttgctattgctgtgagaagtatccaatggagttatgcaattttctggtccaattcagttgcacaaccaggggtcttggagtggggtgatgggttctacaatggagatattaaaactcgaaaaactgtacaatctgtcgaattgaatcaagatcagctgggattgcagagaagtgatcaattgagagaactttatgagtctctttcacttggtgaaaccaacacacaagctaaaaggcctactgctgcattatcaccagaagacctcactgatgctgagtggtttttcttggtttgcatgtctttcatattcaatattggccaagggttgcctggaagaacattagcacgaaatcaagcagtatggctatgcaacgctcatcgtgcggacaccaaagttttctcgcgttctttgcttgcaaagagtgcgtcaattcagacagttgtgtgctttccatattcagaaggtgtagttgagctgggagcaacagagctagtaccggaggatttgaatctaatccagcatataaaaacttcattcttggacagtcctgccaccgttcccaagattcccaactatgtctccaacagtattacaaacaacaatgacctcatttgtgaagcgcttgaacatgctaatataccagaaaacgatcttgatcagcttttgaattgtccagacacgaacatatgttctcctgataacagtttggatgactttgcagacaatttactcatagacgaatcgaatttggcagaaggcatcaatggggaggttcctcaaacacaaagctggcctttcatggatgatgcaatcagcaattgtctcaatagttctatgaattctagtgactgtatatctcaaactcatgaaaatctagagtcttttgctccactttctgatggaaaagggccaccggagacgaataattgtatgcacagcactcaaaaatgcaatcagcagatagaaaacacgggtgtccaaggcgatgaggtccattatcaaggggtactttccaatcttttgaagagttcccatcagttggttcttggtccctacttcagaaatgggaatagagaatcaagcttcgttagttggaacaaggatggatcgtcgggtactcatgttccccgaagcggaacctcacaaagatttctgaagaaagtactttttgaagtagctagaatgcatgaaaactccaggcttgatgctggtaaacaaaagggcaacagtgactgccttgcaaagccaacggctgatgaaattgatagaaaccacgtcttgtcagagagaaaacgcagagagaaaataaacgaacggtttatgattcttgcatccctagtcccatccggtggcaaggttgacaaagtatcaatactagaccatacaatagattacttgagagggcttgagaggaaagtcgacgagctggaatctaacaaaatggtaaagggccgggggcgggaatcaactacaaaaactaaactacacgatgccattgagaggacctctgataattatggcgcaacaaggacaagtaacgtcaagaaaccgttgacaaacaagagaaaggcttctgatacggacaagattggagccgtaaatagcagaggtcgattgaaagattccttaacagataatataactgtgaacattacaaacaaggatgtgttgattgtcgtgacttgttcttccaaggagtttgtattgcttgaagtgatggaagccgtaagacgactaagtttggattccgaaactgttcaatcttccaacagagatggaatgatatctattaccataaaagccaagtgcaagggattgaaggttgcatcagcaagtgtgatcaaacaagctcttcagaaagttactatgaagtcttgacccgggctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatgggccccccctcgaagtcgacggtattgataagcttgatatcgaattcctgcagcccgggggatccgtcgagctgcaagtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattttagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgatgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagacatcatcttaattctcgattagggtttcatagacatcatccgatttgttcaaataatttgagttttgtcgaataattactcttcgatttgtgatttctatctaggtctggtgttagtttctagtttgtgcgatcgaatttgtcgattaatctgagtttttctgattaacaggcccgggatgggaagagcgccatgttgcgagaaggtcggtatcaagagagggcggtggacggcggaggaggaccagattctctccaactacattcaatccaacggtgaaggttcttggagatctctccccaaaaatgccggattaaaaaggtgtggaaagagctgtagattgagatggataaactatctaagatcagacctcaagcgtggaaacataactccagaagaagaagaactcgttgttaaattgcattccactttgggaaacaggtggtcactaatcgcgggtcatctaccagggagaacagacaacgaaataaaaaattattggaactctcatctcagccgtaaactccacaacttcattaggaagccatccatctctcaagacgtctccgccgtaatcatgacgaacgcttcttcagcgccaccgccgccgcaggcaaaacgcagacttgggagaacgggtaggtccgctatgaaaccaaaaatccacagaacaaaaactcgtaaaacgaagaaaacgtctgcaccaccggagcctaacgccgatgtagctggggctgataaagaagcattaatggtggagtcaagtggagccgaggctgagctaggacgaccatgtgactactatggagatgattgtaacaaaaatctcatgagcattaatggcgataatggagttttaacgtttgatgatgatatcatcgatcttttgttggacgagtcagatcctggccacttgtacacaaacacaacgtgcggtggtgatggggagttgcataacataagagactctgaaggagccagagggttctcggatacttggaaccaagggaatctcgactgtcttcttcagtcttgtccatcggtggagtcgtttctcaactacgaccaccaagttaacgatgcgtcgacggatgagtttatcgattgggattgtgtttggcaagaaggtagtgataataatctttggcatgagaaagagaatcccgactcaatggtctcgtggcttttagacggtgatgatgaggccacgatcgggaatagtaattgtgagaactttggagaaccgttagatcatgacgacgaaagcgctttggtcgcttggcttctgtcatgacccgggtaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctggatccactagtgtcgagctgcaagtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattttagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgatgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagacatcatcttaattctcgattagggtttcatagacatcatccgatttgttcaaataatttgagttttgtcgaataattactcttcgatttgtgatttctatctaggtctggtgttagtttctagtttgtgcgatcgaatttgtcgattaatctgagtttttctgattaacaggcccgggatggaaaagaattgtcgtggagtgagaaaaggtacttggaccaaagaagaagacactctcttgaggcaatgtatagaagagtatggtgaagggaaatggcatcaagttccacacagagcagggttgaaccggtgtaggaagagttgcaggctgaggtggttgaattatctgaggccaaatatcaaaagaggtcggttttcgagagatgaagtggacctaattgtgaggcttcataagctgttgggtaacaaatggtcgctgattgctggtagaattcctggaaggacagctaatgacgtgaagaacttttggaatactcatgtggggaagaatttaggcgaggatggagaacgatgccggaaaaatgttatgaacacaaaaaccattaagctgactaatatcgtaagaccccgagctcggaccttcaccggattgcacgttacttggccgagagaagtcggaaaaaccgatgaattttcaaatgtccggttaacaactgatgagattccagattgtgagaagcaaacgcaattttacaatgatgttgcgtcgccacaagatgaagttgaagactgcattcagtggtggagtaagttgctagaaacaacggaggatggggaattaggaaacctattcgaggaggcccaacaaattggaaattaacccgggtaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctactagttctagagcggccgcaccgcggtggagctcggcactggccgtcgttttacaacgtcgtgactgggagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 2 - DNA, 13768 bp, synthetic construct
  source 1..13768: mol_type = other DNA
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgacatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacctcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcggggaggcgcgcctgatcagcttgcatgccggtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatggcaagaccggcaacaggattcaatcttaagaaactttattgccaaatgtttgaacgatctgcttgactctagctagagtccgaaccccagagtcccgctcagaagaactcgtcaagaaggcgatagaaggctatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcgtcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcggtgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctcatgtataggcgcgcctatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggcgtgtggaattgtgagcggataacaatttcacacaggaaacagctatgaccatgattacggtaccgtcgagctgcaagtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattttagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgatgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagacatcatcttaattctcgattagggtttcatagacatcatccgatttgttcaaataatttgagttttgtcgaataattactcttcgatttgtgatttctatctaggtctggtgttagtttctagtttgtgcgatcgaatttgtcgattaatctgagtttttctgattaacaggcccgggatgtcgaagacggtggaggatcgggcagcgcagcgggcaaaggggccggcaacagtgctggccatcggcacggctacgccggccaatgtggtgtaccagaccgattatccggactactacttcagggtcaccaagagcgagcatatgaccaaactcaagaacaagtttcaacgcatgtgcgacaggtcgacgataaagaagaggtacatggttttgacagaggagctgctagagaagaatctgagtttgtgcacctacatggaaccctccctcgacgcccgccaagacattctcgtgccggaggtccccaagctcggcaaggaggccgccgacgaggccatcgccgaatggggacgccccaagtcggaaatcacccacctcatcttttgcactacctgcggcgtcgacatgcccggcgccgactaccagctcaccaagctcctcggtctccgctcctccgtccgtcgcaccatgctctatcagcagggatgctttggcggaggcaccgttctccgcctcgccaaggacctcgccgagaacaacgctggtgcccgcgtcctcgtcgtctgctccgagatcaccactgccgtcaacttccgagggccttccgacacccacctcgacttattggtcggcttagccctgttcggcgacggtgcggccgcggtcatagtcggtgcggatccagatcctaccctcgagcggccgctctttcaaatcgtatctggagcacagacgattctaccggactcggagggggccatcaacggccatctccgggaggtggggctaaccatccgcctactcaaggacgtacctgggcttgtgtcgatgaacattgagaagtgcctcatggaggcgtttgcaccgatgggcatccacgactggaactccatcttttggatagcccatcccggggggcccaccatactagaccaagtggaggccaagctgggtctaaaggaggagaagctcaagtcgacgagggctgttctgagggagtatggcaacatgtctagcgcctgtgtcttgttcatactggacgaggtaaggaagaggagcatggaggaggggaagacgacaaccggtgaggggttcgattggggagttctattcggctttgggcctggcttcacagtggagaccgtcgtcttgcacagcatgcccatccccaaagccgatgaaggcagataacccgggtaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctggtaccgggccccccctcgaagtcgacggtattgataagcttgatatcgaattcctgcagcccggggatcctgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggctatcgttcaagatgcctctaccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgcccgggatggagcaagctgtgttcaaagaccaatccccaagcagggatgatattgatgaagagctctttcaatctgctctatatcttagcactgcggttgtcaccgtgccggcggcaatcatggctgcaaatgaccttgacgtgctgcagataattgccaaagctggcccaggtgctcacctatctccgacagagattgtcagccaccttcccacccgtaaccctaatgccgcggcggcgcttcaccggatactccgagtactagccagccactccatccttgaatgctcgtcgagatgcgagggcgaggcaaaatatggattaaggccggtgtgcaagttctttctcaatgataaggatggtgtctccttgaatgccatgccatccttcgttcaaagtagagtttttatagatagctggcaatatatgaaagatgctgttcttgagggggtagtcccctttgagaaagcctatggtatgcctttttatcagtttcaagcagtgaacaccaaattcaaagaaaccttcgccaaagccatggctgctcactcaactttggtagtaaaaaagatgcttgacacatacaatgggtttgagggactcactgagttgatggatgttgctggtggaaccggttccaccctcaacctcattgtctccaaatacccacaaatcaaaggcacaaactttgatctcaaacatgtcattgaggccgcaccaaactaccctggggtgaagcatttgagtggggacatgtttgatagcattccaagtgcaaagaacattattatgaagtggatactacataattggagcgacgagcactgtgtaaaactcctcaagaactgctacacttccttaccagaatttgggaagttgattgtggttgattccattgtgggtgaggatgttgatgctggtttgacgacaacaaatgtctttggatgcgacttcacaatgctaactttcttccccaatgcaaaagagaggacccgtgaagaattccaagacctggccaaagctagtggcttctcaacgttcaaaccgatctgctgcgcctatggcgtgtgggttatggaatttcacaaataacccgggctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatggatccactagttctagagcggccgcaccgcggtggagctcggcactggccgtcgttttacaacgtcgtgactgggagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 3 - DNA, 14107 bp, synthetic construct
  source 1..14107: mol_type = other DNA
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgacatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacctcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcggggaggcgcgcctgatcagcttgcatgccggtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatggcaagaccggcaacaggattcaatcttaagaaactttattgccaaatgtttgaacgatctgcttgactctagctagagtccgaaccccagagtcccgctcagaagaactcgtcaagaaggcgatagaaggctatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcgtcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcggtgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctcatgtataggcgcgcctatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggcgtgtggaattgtgagcggataacaatttcacacaggaaacagctatgaccatgattacggtaccgggcccgaattgacgcgtattgggattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggctatcgttcaagatgcctctaccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgcccgggatggctactggtatccaaaaccaaaagatagtgcctgagaatttgaggaagcaacttgctattgctgtgagaagtatccaatggagttatgcaattttctggtccaattcagttgcacaaccaggggtcttggagtggggtgatgggttctacaatggagatattaaaactcgaaaaactgtacaatctgtcgaattgaatcaagatcagctgggattgcagagaagtgatcaattgagagaactttatgagtctctttcacttggtgaaaccaacacacaagctaaaaggcctactgctgcattatcaccagaagacctcactgatgctgagtggtttttcttggtttgcatgtctttcatattcaatattggccaagggttgcctggaagaacattagcacgaaatcaagcagtatggctatgcaacgctcatcgtgcggacaccaaagttttctcgcgttctttgcttgcaaagagtgcgtcaattcagacagttgtgtgctttccatattcagaaggtgtagttgagctgggagcaacagagctagtaccggaggatttgaatctaatccagcatataaaaacttcattcttggacagtcctgccaccgttcccaagattcccaactatgtctccaacagtattacaaacaacaatgacctcatttgtgaagcgcttgaacatgctaatataccagaaaacgatcttgatcagcttttgaattgtccagacacgaacatatgttctcctgataacagtttggatgactttgcagacaatttactcatagacgaatcgaatttggcagaaggcatcaatggggaggttcctcaaacacaaagctggcctttcatggatgatgcaatcagcaattgtctcaatagttctatgaattctagtgactgtatatctcaaactcatgaaaatctagagtcttttgctccactttctgatggaaaagggccaccggagacgaataattgtatgcacagcactcaaaaatgcaatcagcagatagaaaacacgggtgtccaaggcgatgaggtccattatcaaggggtactttccaatcttttgaagagttcccatcagttggttcttggtccctacttcagaaatgggaatagagaatcaagcttcgttagttggaacaaggatggatcgtcgggtactcatgttccccgaagcggaacctcacaaagatttctgaagaaagtactttttgaagtagctagaatgcatgaaaactccaggcttgatgctggtaaacaaaagggcaacagtgactgccttgcaaagccaacggctgatgaaattgatagaaaccacgtcttgtcagagagaaaacgcagagagaaaataaacgaacggtttatgattcttgcatccctagtcccatccggtggcaaggttgacaaagtatcaatactagaccatacaatagattacttgagagggcttgagaggaaagtcgacgagctggaatctaacaaaatggtaaagggccgggggcgggaatcaactacaaaaactaaactacacgatgccattgagaggacctctgataattatggcgcaacaaggacaagtaacgtcaagaaaccgttgacaaacaagagaaaggcttctgatacggacaagattggagccgtaaatagcagaggtcgattgaaagattccttaacagataatataactgtgaacattacaaacaaggatgtgttgattgtcgtgacttgttcttccaaggagtttgtattgcttgaagtgatggaagccgtaagacgactaagtttggattccgaaactgttcaatcttccaacagagatggaatgatatctattaccataaaagccaagtgcaagggattgaaggttgcatcagcaagtgtgatcaaacaagctcttcagaaagttactatgaagtcttgacccgggctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatgggccccccctcgaagtcgacggtattgataagcttgatatcgaattcctgcagcccgggggatccactagtgtcgagctgcaagtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattttagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgatgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagacatcatcttaattctcgattagggtttcatagacatcatccgatttgttcaaataatttgagttttgtcgaataattactcttcgatttgtgatttctatctaggtctggtgttagtttctagtttgtgcgatcgaatttgtcgattaatctgagtttttctgattaacaggcccgggatggaaaagaattgtcgtggagtgagaaaaggtacttggaccaaagaagaagacactctcttgaggcaatgtatagaagagtatggtgaagggaaatggcatcaagttccacacagagcagggttgaaccggtgtaggaagagttgcaggctgaggtggttgaattatctgaggccaaatatcaaaagaggtcggttttcgagagatgaagtggacctaattgtgaggcttcataagctgttgggtaacaaatggtcgctgattgctggtagaattcctggaaggacagctaatgacgtgaagaacttttggaatactcatgtggggaagaatttaggcgaggatggagaacgatgccggaaaaatgttatgaacacaaaaaccattaagctgactaatatcgtaagaccccgagctcggaccttcaccggattgcacgttacttggccgagagaagtcggaaaaaccgatgaattttcaaatgtccggttaacaactgatgagattccagattgtgagaagcaaacgcaattttacaatgatgttgcgtcgccacaagatgaagttgaagactgcattcagtggtggagtaagttgctagaaacaacggaggatggggaattaggaaacctattcgaggaggcccaacaaattggaaattaacccgggtaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctactagttctagagcggccgcaccgcggtggagctcggcactggccgtcgttttacaacgtcgtgactgggagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 4 - DNA, 10656 bp, synthetic construct
  source 1..10656: mol_type = other DNA
gtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgccagtgccaagctaattcgaggggatttaaattggtcgatctggttgtactgaggacttctacaagaaggacagctctgtttgaggtcggattgggatcgcttgaaaagcttgttctgttacgtttgatccctccaccatgagcaacgagctgaattgaaactcagaaaggtcttgctgcatcaactgatgcaggtggcaaagacactgaagggtcgatgaggtgcagagcgaatcgacagcagctgtaagggttcctcttccttctcctcctcctcctcctcttccttcgcagcaccgcattatgcttgaattgtgttgcgctccggcgtcactcccatgaacatgaaaacacggaccagcaccgaacctacgttttcatcgacgatttagtgttgaaacctcacctataatgacttcaatggctttgcaagtgcattagtagtgattacatacaataattttaaatctcaattggcactaaataatgtcaacaagattaaattactaccaaatccagttgggttacaaaatgaaacaaaagcatctgacaatctcaaacatgaaaagactcgtgcacatggtctcaaaaaagttagaaggtaatgattgagcaagaaaatactttatcacattcattgatgattgagtggaatctgaagggagatagcataaaggtcttacataacatttattgataattgagtacaattgaagtaaagcctggacaacaacttcaatacatctccaccgcggtggcggccgctctagaactagtggatcccccggggtcgagctgcaagtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattttagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgatgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagacatcatcttaattctcgattagggtttcatagacatcatccgatttgttcaaataatttgagttttgtcgaataattactcttcgatttgtgatttctatctaggtctggtgttagtttctagtttgtgcgatcgaatttgtcgattaatctgagtttttctgattaacaggcccgggatgtcgaagacggtggaggatcgggcagcgcagcgggcaaaggggccggcaacagtgctggccatcggcacggctacgccggccaatgtggtgtaccagaccgattatccggactactacttcagggtcaccaagagcgagcatatgaccaaactcaagaacaagtttcaacgcatgtgcgacaggtcgacgataaagaagaggtacatggttttgacagaggagctgctagagaagaatctgagtttgtgcacctacatggaaccctccctcgacgcccgccaagacattctcgtgccggaggtccccaagctcggcaaggaggccgccgacgaggccatcgccgaatggggacgccccaagtcggaaatcacccacctcatcttttgcactacctgcggcgtcgacatgcccggcgccgactaccagctcaccaagctcctcggtctccgctcctccgtccgtcgcaccatgctctatcagcagggatgctttggcggaggcaccgttctccgcctcgccaaggacctcgccgagaacaacgctggtgcccgcgtcctcgtcgtctgctccgagatcaccactgccgtcaacttccgagggccttccgacacccacctcgacttattggtcggcttagccctgttcggcgacggtgcggccgcggtcatagtcggtgcggatccagatcctaccctcgagcggccgctctttcaaatcgtatctggagcacagacgattctaccggactcggagggggccatcaacggccatctccgggaggtggggctaaccatccgcctactcaaggacgtacctgggcttgtgtcgatgaacattgagaagtgcctcatggaggcgtttgcaccgatgggcatccacgactggaactccatcttttggatagcccatcccggggggcccaccatactagaccaagtggaggccaagctgggtctaaaggaggagaagctcaagtcgacgagggctgttctgagggagtatggcaacatgtctagcgcctgtgtcttgttcatactggacgaggtaaggaagaggagcatggaggaggggaagacgacaaccggtgaggggttcgattggggagttctattcggctttgggcctggcttcacagtggagaccgtcgtcttgcacagcatgcccatccccaaagccgatgaaggcagataacccgggtaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctggtaccgggccccccctcgaagtcgacggtattgataagcttgatatcgaattcctgcagcccggggatcctgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggctatcgttcaagatgcctctaccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgcccgggatggagcaagctgtgttcaaagaccaatccccaagcagggatgatattgatgaagagctctttcaatctgctctatatcttagcactgcggttgtcaccgtgccggcggcaatcatggctgcaaatgaccttgacgtgctgcagataattgccaaagctggcccaggtgctcacctatctccgacagagattgtcagccaccttcccacccgtaaccctaatgccgcggcggcgcttcaccggatactccgagtactagccagccactccatccttgaatgctcgtcgagatgcgagggcgaggcaaaatatggattaaggccggtgtgcaagttctttctcaatgataaggatggtgtctccttgaatgccatgccatccttcgttcaaagtagagtttttatagatagctggcaatatatgaaagatgctgttcttgagggggtagtcccctttgagaaagcctatggtatgcctttttatcagtttcaagcagtgaacaccaaattcaaagaaaccttcgccaaagccatggctgctcactcaactttggtagtaaaaaagatgcttgacacatacaatgggtttgagggactcactgagttgatggatgttgctggtggaaccggttccaccctcaacctcattgtctccaaatacccacaaatcaaaggcacaaactttgatctcaaacatgtcattgaggccgcaccaaactaccctggggtgaagcatttgagtggggacatgtttgatagcattccaagtgcaaagaacattattatgaagtggatactacataattggagcgacgagcactgtgtaaaactcctcaagaactgctacacttccttaccagaatttgggaagttgattgtggttgattccattgtgggtgaggatgttgatgctggtttgacgacaacaaatgtctttggatgcgacttcacaatgctaactttcttccccaatgcaaaagagaggacccgtgaagaattccaagacctggccaaagctagtggcttctcaacgttcaaaccgatctgctgcgcctatggcgtgtgggttatggaatttcacaaataacccgggctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccgggctgcaggaattagcttatcgataccgtcgacctcgagggggggcccggtaccaagcttgctagcgaattcgatatcccgggcgcgcggtaccataacttcgtataatgtatgctatacgaagttatcctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccaagcatggagaaccgacctgcaggcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcagggactcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcggacggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccataacttcgtataatgtatgctatacgaagttatgcatgcgatagcagctgtccttctcaagtggaaaggtagtgtgaataggtggagtgagggaagagcttctcctcaagtgctctgacaactttagggtttatttgcaggaagctaccaagtgttgaatgaccaagccatcgctttatgattgcaagagtactccctgacacgcgtggaacaggggattaagtgaatgggagggaatgcgtttctgagaggaatctcatgttctactatgagaatgtagagaaaacaagaggcgtccgtgttgcacttgtgtgggtgcatgtcgtacttgcaagttttgtagaggaaaacttaagttacttatgtttcgcaagagttttaggtttcgagggatcgccactgccagatttagacacacacttttcaaagagtgagagggggcgaggttgaggggagaacgctggcgcgcacagggggcgcctgatggctgatcggcctcaagcacatgtaacttttcatgaaattcgtctggaagtgaaagagccgaagtacaaaagcgtgagtatcatcttaagcggagctggagataagggagggggcgaaagcggggaagaatgctgagtgtagtgcgggtgcacctcccgaccgacattcccattgtaggctgcgagttgtcggcctatgtgctgttgcgacggggagattcgagtctttctcctgaagatgttaccgaggcatcgtctacggatggctattttctccagtgtcgatggtatctgtccttacttctaaacttttctttttgtttattgtggaacattcatgctaacgttcttcttgaaagacgacagtccgtgagttgaatttaaatcccatctcgagatctagcttggc

SEQ ID NO: 5 - DNA, 11571 bp, synthetic construct
  source 1..11571: mol_type = other DNA
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgacatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacctcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcggggaggcgcgcctgatcagcttgcatgccggtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatggcaagaccggcaacaggattcaatcttaagaaactttattgccaaatgtttgaacgatctgcttgactctagctagagtccgaaccccagagtcccgctcagaagaactcgtcaagaaggcgatagaaggctatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcgtcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcggtgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctcatgtataggcgcgcctatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggcgtgtggaattgtgagcggataacaatttcacacaggaaacagctatgaccatgattacggtaccgggcccgtcgagctgcaagtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattttagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgatgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagacatcatcttaattctcgattagggtttcatagacatcatccgatttgttcaaataatttgagttttgtcgaataattactcttcgatttgtgatttctatctaggtctggtgttagtttctagtttgtgcgatcgaatttgtcgattaatctgagtttttctgattaacaggcccgggatgggaagagcgccatgttgcgagaaggtcggtatcaagagagggcggtggacggcggaggaggaccagattctctccaactacattcaatccaacggtgaaggttcttggagatctctccccaaaaatgccggattaaaaaggtgtggaaagagctgtagattgagatggataaactatctaagatcagacctcaagcgtggaaacataactccagaagaagaagaactcgttgttaaattgcattccactttgggaaacaggtggtcactaatcgcgggtcatctaccagggagaacagacaacgaaataaaaaattattggaactctcatctcagccgtaaactccacaacttcattaggaagccatccatctctcaagacgtctccgccgtaatcatgacgaacgcttcttcagcgccaccgccgccgcaggcaaaacgcagacttgggagaacgggtaggtccgctatgaaaccaaaaatccacagaacaaaaactcgtaaaacgaagaaaacgtctgcaccaccggagcctaacgccgatgtagctggggctgataaagaagcattaatggtggagtcaagtggagccgaggctgagctaggacgaccatgtgactactatggagatgattgtaacaaaaatctcatgagcattaatggcgataatggagttttaacgtttgatgatgatatcatcgatcttttgttggacgagtcagatcctggccacttgtacacaaacacaacgtgcggtggtgatggggagttgcataacataagagactctgaaggagccagagggttctcggatacttggaaccaagggaatctcgactgtcttcttcagtcttgtccatcggtggagtcgtttctcaactacgaccaccaagttaacgatgcgtcgacggatgagtttatcgattgggattgtgtttggcaagaaggtagtgataataatctttggcatgagaaagagaatcccgactcaatggtctcgtggcttttagacggtgatgatgaggccacgatcgggaatagtaattgtgagaactttggagaaccgttagatcatgacgacgaaagcgctttggtcgcttggcttctgtcatgacccgggtaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctgggccccccctcgaagtcgacggtattgataagcttgatatcgaattcctgcagcccgggggatccactagttctagagcggccgcaccgcggtggagctcggcactggccgtcgttttacaacgtcgtgactgggagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 6 - DNA, 22272 bp, synthetic construct
  source 1..22272: mol_type = other DNA
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgacatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaacctcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcggggaggcgcgcctgatcagcttgcatgccggtcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatggcaagaccggcaacaggattcaatcttaagaaactttattgccaaatgtttgaacgatctgcttgactctagctagagtccgaaccccagagtcccgctcagaagaactcgtcaagaaggcgatagaaggctatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcgtcgccgtgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcggtgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctcatgtataggcgcgcctatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggcgtgtggaattgtgagcggataacaatttcacacaggaaacagctatgaccatgattacggtaccgtcgagctgcaagtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattttagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgatgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagacatcatcttaattctcgattagggtttcatagacatcatccgatttgttcaaataatttgagttttgtcgaataattactcttcgatttgtgatttctatctaggtctggtgttagtttctagtttgtgcgatcgaatttgtcgattaatctgagtttttctgattaacaggcccgggatgtcgaagacggtggaggatcgggcagcgcagcgggcaaaggggccggcaacagtgctggccatcggcacggctacgccggccaatgtggtgtaccagaccgattatccggactactacttcagggtcaccaagagcgagcatatgaccaaactcaagaacaagtttcaacgcatgtgcgacaggtcgacgataaagaagaggtacatggttttgacagaggagctgctagagaagaatctgagtttgtgcacctacatggaaccctccctcgacgcccgccaagacattctcgtgccggaggtccccaagctcggcaaggaggccgccgacgaggccatcgccgaatggggacgccccaagtcggaaatcacccacctcatcttttgcactacctgcggcgtcgacatgcccggcgccgactaccagctcaccaagctcctcggtctccgctcctccgtccgtcgcaccatgctctatcagcagggatgctttggcggaggcaccgttctccgcctcgccaaggacctcgccgagaacaacgctggtgcccgcgtcctcgtcgtctgctccgagatcaccactgccgtcaacttccgagggccttccgacacccacctcgacttattggtcggcttagccctgttcggcgacggtgcggccgcggtcatagtcggtgcggatccagatcctaccctcgagcggccgctctttcaaatcgtatctggagcacagacgattctaccggactcggagggggccatcaacggccatctccgggaggtggggctaaccatccgcctactcaaggacgtacctgggcttgtgtcgatgaacattgagaagtgcctcatggaggcgtttgcaccgatgggcatccacgactggaactccatcttttggatagcccatcccggggggcccaccatactagaccaagtggaggccaagctgggtctaaaggaggagaagctcaagtcgacgagggctgttctgagggagtatggcaacatgtctagcgcctgtgtcttgttcatactggacgaggtaaggaagaggagcatggaggaggggaagacgacaaccggtgaggggttcgattggggagttctattcggctttgggcctggcttcacagtggagaccgtcgtcttgcacagcatgcccatccccaaagccgatgaaggcagataacccgggtaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctggtaccgggccccccctcgaagtcgacggtattgataagctttgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggctatcgttcaagatgcctctaccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgcccgggatggctactggtatccaaaaccaaaagatagtgcctgagaatttgaggaagcaacttgctattgctgtgagaagtatccaatggagttatgcaattttctggtccaattcagttgcacaaccaggggtcttggagtggggtgatgggttctacaatggagatattaaaactcgaaaaactgtacaatctgtcgaattgaatcaagatcagctgggattgcagagaagtgatcaattgagagaactttatgagtctctttcacttggtgaaaccaacacacaagctaaaaggcctactgctgcattatcaccagaagacctcactgatgctgagtggtttttcttggtttgcatgtctttcatattcaatattggccaagggttgcctggaagaacattagcacgaaatcaagcagtatggctatgcaacgctcatcgtgcggacaccaaagttttctcgcgttctttgcttgcaaagagtgcgtcaattcagacagttgtgtgctttccatattcagaaggtgtagttgagctgggagcaacagagctagtaccggaggatttgaatctaatccagcatataaaaacttcattcttggacagtcctgccaccgttcccaagattcccaactatgtctccaacagtattacaaacaacaatgacctcatttgtgaagcgcttgaacatgctaatataccagaaaacgatcttgatcagcttttgaattgtccagacacgaacatatgttctcctgataacagtttggatgactttgcagacaatttactcatagacgaatcgaatttggcagaaggcatcaatggggaggttcctcaaacacaaagctggcctttcatggatgatgcaatcagcaattgtctcaatagttctatgaattctagtgactgtatatctcaaactcatgaaaatctagagtcttttgctccactttctgatggaaaagggccaccggagacgaataattgtatgcacagcactcaaaaatgcaatcagcagatagaaaacacgggtgtccaaggcgatgaggtccattatcaaggggtactttccaatcttttgaagagttcccatcagttggttcttggtccctacttcagaaatgggaatagagaatcaagcttcgttagttggaacaaggatggatcgtcgggtactcatgttccccgaagcggaacctcacaaagatttctgaagaaagtactttttgaagtagctagaatgcatgaaaactccaggcttgatgctggtaaacaaaagggcaacagtgactgccttgcaaagccaacggctgatgaaattgatagaaaccacgtcttgtcagagagaaaacgcagagagaaaataaacgaacggtttatgattcttgcatccctagtcccatccggtggcaaggttgacaaagtatcaatactagaccatacaatagattacttgagagggcttgagaggaaagtcgacgagctggaatctaacaaaatggtaaagggccgggggcgggaatcaactacaaaaactaaactacacgatgccattgagaggacctctgataattatggcgcaacaaggacaagtaacgtcaagaaaccgttgacaaacaagagaaaggcttctgatacggacaagattggagccgtaaatagcagaggtcgattgaaagattccttaacagataatataactgtgaacattacaaacaaggatgtgttgattgtcgtgacttgttcttccaaggagtttgtattgcttgaagtgatggaagccgtaagacgactaagtttggattccgaaactgttcaatcttccaacagagatggaatgatatctattaccataaaagccaagtgcaagggattgaaggttgcatcagcaagtgtgatcaaacaagctcttcagaaagttactatgaagtcttgacccgggctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatgggccccccctcgaagtcgacggtattgataagcttgatatcgaattcctgcagcccgggggatccgtcgagctgcaagtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattttagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgatgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagacatcatcttaattctcgattagggtttcatagacatcatccgatttgttcaaataatttgagttttgtcgaataattactcttcgatttgtgatttctatctaggtctggtgttagtttctagtttgtgcgatcgaatttgtcgattaatctgagtttttctgattaacaggcccgggatgggaagagcgccatgttgcgagaaggtcggtatcaagagagggcggtggacggcggaggaggaccagattctctccaactacattcaatccaacggtgaaggttcttggagatctctccccaaaaatgccggattaaaaaggtgtggaaagagctgtagattgagatggataaactatctaagatcagacctcaagcgtggaaacataactccagaagaagaagaactcgttgttaaattgcattccactttgggaaacaggtggtcactaatcgcgggtcatctaccagggagaacagacaacgaaataaaaaattattggaactctcatctcagccgtaaactccacaacttcattaggaagccatccatctctcaagacgtctccgccgtaatcatgacgaacgcttcttcagcgccaccgccgccgcaggcaaaacgcagacttgggagaacgggtaggtccgctatgaaaccaaaaatccacagaacaaaaactcgtaaaacgaagaaaacgtctgcaccaccggagcctaacgccgatgtagctggggctgataaagaagcattaatggtggagtcaagtggagccgaggctgagctaggacgaccatgtgactactatggagatgattgtaacaaaaatctcatgagcattaatggcgataatggagttttaacgtttgatgatgatatcatcgatcttttgttggacgagtcagatcctggccacttgtacacaaacacaacgtgcggtggtgatggggagttgcataacataagagactctgaaggagccagagggttctcggatacttggaaccaagggaatctcgactgtcttcttcagtcttgtccatcggtggagtcgtttctcaactacgaccaccaagttaacgatgcgtcgacggatgagtttatcgattgggattgtgtttggcaagaaggtagtgataataatctttggcatgagaaagagaatcccgactcaatggtctcgtggcttttagacggtgatgatgaggccacgatcgggaatagtaattgtgagaactttggagaaccgttagatcatgacgacgaaagcgctttggtcgcttggcttctgtcatgacccgggtaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctggatccactagtgtcgagctgcaagtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattttagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgatgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagacatcatcttaattctcgattagggtttcatagacatcatccgatttgttcaaataatttgagttttgtcgaataattactcttcgatttgtgatttctatctaggtctggtgttagtttctagtttgtgcgatcgaatttgtcgattaatctgagtttttctgattaacaggcccgggatggaaaagaattgtcgtggagtgagaaaaggtacttggaccaaagaagaagacactctcttgaggcaatgtatagaagagtatggtgaagggaaatggcatcaagttccacacagagcagggttgaaccggtgtaggaagagttgcaggctgaggtggttgaattatctgaggccaaatatcaaaagaggtcggttttcgagagatgaagtggacctaattgtgaggcttcataagctgttgggtaacaaatggtcgctgattgctggtagaattcctggaaggacagctaatgacgtgaagaacttttggaatactcatgtggggaagaatttaggcgaggatggagaacgatgccggaaaaatgttatgaacacaaaaaccattaagctgactaatatcgtaagaccccgagctcggaccttcaccggattgcacgttacttggccgagagaagtcggaaaaaccgatgaattttcaaatgtccggttaacaactgatgagattccagattgtgagaagcaaacgcaattttacaatgatgttgcgtcgccacaagatgaagttgaagactgcattcagtggtggagtaagttgctagaaacaacggaggatggggaattaggaaacctattcgaggaggcccaacaaattggaaattaacccgggtaagaaatgcctgtaatgtcccacaaatgtagaaaatggttcgtacctttgtccaagcattgatatgtctgatgagaggaaactgcaagatactgagcttggtttaacgaaggagaggcagtttcttccttccaaagcatttcatttgacaatgccttgatcatcttaagtagagtttctgttgtggaaagtttgaaactttgaagaaacgactctcaagtaaattgatgatcacaagtgaaagtgtatgttacataagtggatatttcaccctttttccatcaatcaaaacatcatatagtaatccattggtttatacaaacatcaaaatacatttacctctgaaatgaggaaaaaaatgcaaagagatttttgaaaatttccaacaaatggagagaaaaaaaaaaaacaaagaattaaatcaatcagtacatttataaaggttatgagacctatatgacacttattggtcaagtggcagccattccatttctagctctaagcttgatatcgaattcctgcagcccggggatcctgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggctatcgttcaagatgcctctaccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacctcgagaattaattctcaacacaacatatacaaaacaaacgaatctcaagcaatcaagcattctacttctattgcagcaatttaaatcatttcttttaaagcaaaagcaattttctgaaaattttcaccatttacgaacgcccgggatggagcaagctgtgttcaaagaccaatccccaagcagggatgatattgatgaagagctctttcaatctgctctatatcttagcactgcggttgtcaccgtgccggcggcaatcatggctgcaaatgaccttgacgtgctgcagataattgccaaagctggcccaggtgctcacctatctccgacagagattgtcagccaccttcccacccgtaaccctaatgccgcggcggcgcttcaccggatactccgagtactagccagccactccatccttgaatgctcgtcgagatgcgagggcgaggcaaaatatggattaaggccggtgtgcaagttctttctcaatgataaggatggtgtctccttgaatgccatgccatccttcgttcaaagtagagtttttatagatagctggcaatatatgaaagatgctgttcttgagggggtagtcccctttgagaaagcctatggtatgcctttttatcagtttcaagcagtgaacaccaaattcaaagaaaccttcgccaaagccatggctgctcactcaactttggtagtaaaaaagatgcttgacacatacaatgggtttgagggactcactgagttgatggatgttgctggtggaaccggttccaccctcaacctcattgtctccaaatacccacaaatcaaaggcacaaactttgatctcaaacatgtcattgaggccgcaccaaactaccctggggtgaagcatttgagtggggacatgtttgatagcattccaagtgcaaagaacattattatgaagtggatactacataattggagcgacgagcactgtgtaaaactcctcaagaactgctacacttccttaccagaatttgggaagttgattgtggttgattccattgtgggtgaggatgttgatgctggtttgacgacaacaaatgtctttggatgcgacttcacaatgctaactttcttccccaatgcaaaagagaggacccgtgaagaattccaagacctggccaaagctagtggcttctcaacgttcaaaccgatctgctgcgcctatggcgtgtgggttatggaatttcacaaataacccgggctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatggatccactagttctagagcggccgcaccgcggtggagctcggcactggccgtcgttttacaacgtcgtgactgggagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg